(19) United States
(12) Patent Application Publication
(10) Pub. No.: US 2005/0069910 A1
(43) Pub. Date: Mar. 31, 2005

(54) Nucleic acid ligands to complex targets

(76) Inventors: John V. Turner, South Australia (AU); Robert James, Wattle Park (AU); Stephen Fitter, Blair Athol (AU); Jan Kazenwadel, Belair (AU); Danielle Horley, Dulwich (AU)

Correspondence Address: BIRCH STEWART KOLASCH & BIRCH, PO BOX 747, FALLS CHURCH, VA, 22040-0747, US

(21) Appl. No.: 10/746,339

(22) Filed: Dec. 29, 2003

Related U.S. Application Data
(63) Continuation-in-part of application No. PCT/AU02/00857, filed on Jun. 28, 2002.

(30) Foreign Application Priority Data
Jun. 29, 2001 (AU) PR 5985
Mar. 28, 2002 (AU) 27754/02

(51) Int. Cl. C12Q 1/68; C12P 19/34

(52) U.S. Cl. 435/6; 435/91.2

(57) ABSTRACT

The present invention relates to a method for isolating a pool of nucleic acid ligands capable of binding to one or more target molecules in a complex mixture.

### FILED OF THE INVENTION

The present invention relates to methods for identifying nucleic acid ligands to specific molecules in complex mixes. The present invention also relates to nucleic acid ligands isolated by such methods.

### BACKGROUND OF THE INVENTION

Many biological and chemical systems are composed of a large number of different interacting molecular species. The manner in which many of these molecules interact with each other determines the properties and functions of the particular system. For example, the function and properties of a particular biological system are due to the many and varied interactions that occur between the proteins, nucleic acids and other molecules that make up the system.

In order to understand how such complex systems function, it is necessary to define the individual interactions that occur between the different molecular species. A first step in defining these interactions is the identification of what molecular species are present in a system, and at what concentration they exist to exert their actions.

An improved understanding of the molecular species present in a complex system, and at what concentrations they exist, is also important in determining how some complex systems undergo a transition from one state to another state. For example, such considerations are important in understanding how the change from a normal state to a diseased state occurs for some cell types. An understanding of the identity and concentration of the molecular species present in a system is also important in terms of diagnosis and prognosis. For example, the transformation of a normal tissue to a pre-malignant tissue, and ultimately to a malignant one, may be able to be identified by an improved understanding of the presence and concentration of the molecular species present at any particular time in the cells of interest.

A powerful tool for the identification of the molecular species present in a complex mixture is the use of probe molecules that have the capacity to bind or interact with a particular molecule of interest. For example, antibodies may be used to identify specific antigens in complex mixtures of antigens. Naturally occurring ligands to a molecule (or engineered variants thereof) may be detectably labelled and used to identify their targets in complex mixtures of receptor molecules. Nucleic acids complementary to another specific nucleic acid may be used to identify and characterise the specific nucleic acid in a complex mixture of nucleic acids.

Accordingly, the generation of ligands with specificity to new or important target molecules is an important tool for research, diagnosis and treatment. However, the generation of new ligands to a specific target molecule is often problematic. In some cases, rational design of new ligands may be effective. In such instances a detailed understanding of the three dimensional structure of the relevant part of the target molecule is usually required. However, many target molecules (for example proteins) have complex structures, making the rational design of new ligands to the molecule difficult.

In some instances it is possible to identify new ligands to a target molecule without knowledge of the structure of the target molecule. In this case, the ability to identify new ligands is usually dependent upon the ability to generate a large number of molecules of different structure, a proportion of which may have the capacity to bind to a target molecule with useful affinity. For example, the generation of antibodies in vivo relies on such a principle. However, for the generation of antibodies specific to a particular target molecule it is usually necessary to first isolate the target antigen and/or screen a large number of monoclonal antibodies for binding to the target antigen. In addition, the use of antibodies as tools is often limited by the capacity to generate and isolate antibodies against specific types of target antigens, and the fact that the generation and testing of antibodies is a time consuming and labour intensive process.

Single stranded nucleic acids also have the capacity to form a multitude of different three dimensional structures. Indeed, single stranded nucleic acids may have a three dimensional structural diversity not unlike proteins. The three dimensional structure adopted by any one single stranded nucleic acid is dependent upon the primary sequence of nucleotides, and ultimately is the result of the numerous types of intra-molecular interactions that occur between atoms present in the molecule and inter-molecular interactions that occur between atoms present in the molecule and the surrounding solvent. The three dimensional structure will also depend upon the kinetics and thermodynamics of folding of any one structure.

Because single stranded nucleic acids have the capacity to form a multitude of different three dimensional structures, they may also be potential ligands to a large variety of different types of target molecules. Single stranded nucleic acids that have the capacity to bind to other target molecules are generally referred to as aptamers. In fact, given the structural diversity possible with single stranded nucleic acids, it may be possible to isolate a single stranded nucleic acid with a useful binding affinity to any molecule of interest.

In this regard, chemical synthesis of nucleic acids allows the generation of a pool of large numbers of single stranded nucleic acids of random nucleotide sequence. If the complexity of the pool of single stranded molecules generated by chemical synthesis is sufficient, it may be possible to isolate a unique nucleic acid ligand to any specific molecule. For example, SELEX (systematic evolution of ligands by exponential enrichment) is a technique that allows the isolation of specific nucleic acid ligands from a starting pool of candidate single stranded nucleic acids. By a process of reiterated steps of binding nucleic acids to a target molecule, isolation of the bound nucleic acids and subsequent amplification, nucleic acid ligands to a specific molecule may be quickly and easily identified.

However, a deficiency in the use of single stranded nucleic acid targets has been the inability to identify and use single stranded nucleic acid ligands to complex mixtures of molecules, as for example are present in cellular extracts. The large number of molecules present in the mixture, and the variety of interactions of varying affinity that are possible between molecules in the mixture and nucleic acid ligands, has made the identification and use of specific nucleic acid ligands to such mixtures problematic.

For example, the isolation of a specific nucleic acid ligand to a specific molecule by a process such as SELEX using purified, or even partially purified targets, does not necessarily result in a nucleic acid ligand that is effective in binding to the specific molecule when that molecule is present in a complex mixture of other potential target molecules. It would be advantageous to isolate nucleic acid ligands that can bind to specific molecules present in complex mixtures. It would also be advantageous to use such ligands to screen for differences in the concentration of specific target molecules between different sets of complex mixtures.

In addition, a further deficiency with the identification of nucleic acid ligands to complex mixtures has been the inability to readily produce a library of different nucleic acid ligands to the complex mixture. For example, it would ultimately be advantageous for many reasons to be able to readily isolate a unique nucleic acid ligand to every biologically significant molecule in a complex mixture.

To produce such a library of nucleic acid ligands by existing SELEX techniques would require the isolation of a specific target molecule present in the complex mixture and the independent isolation of a nucleic acid ligand to that specific molecule. In such a way, by repeating this process for each newly isolated molecule present in the complex mixture, a library of nucleic acid ligands to a number of different molecules in the complex mixture could be built up. However, not only is such a sequential manner of isolating nucleic acid ligands laborious and time consuming, the ligands so isolated may not be effective in binding to their specific target molecules, when those molecules are present in a complex mixture of other molecules.

The present invention relates to methods for the isolation of nucleic acid ligands that are capable of binding to target molecules present in complex mixtures.

### SUMMARY OF THE INVENTION

The present invention provides a method for isolating a nucleic acid ligand capable of binding to a target molecule in a complex mixture, the method including the steps of:
(a) providing a pool of candidate nucleic acid ligands;
(b) providing a pool of target molecules;
(c) allowing the nucleic acid ligands to bind to the target molecules;
(d) isolating nucleic acid ligands bound to the target molecules;
(e) amplifying the isolated nucleic acid ligands;
(f) reiterating steps (a) to (e) using the amplified nucleic acid ligands as the pool of candidate nucleic acid ligands, wherein the steps are reiterated until a final pool of nucleic acid ligands is obtained with a desired level of binding specificity to the pool of target molecules; and
(g) isolating a specific nucleic acid ligand from the final pool of nucleic acid ligands, wherein the specific nucleic acid ligand is capable of binding to a target molecule in a complex mixture.

The present invention also provides a method for isolating a pool of nucleic acid ligands capable of binding to one or more target molecules in a complex mixture, the method including the steps of:
(a) providing a pool of candidate nucleic acid ligands;
(b) providing a first pool of target molecules;
(c) providing a second pool of target molecules, wherein the second pool of target molecules may be isolated from the first pool of target molecules, and wherein the second pool of target molecules differs from the first pool of target molecules in that one or more of the target molecules in the second pool is present at a higher concentration than that present in the first pool of target molecules;
(d) allowing the nucleic acid ligands to bind to the first and second pools of target molecules, wherein the first and second pool of target molecules are in the presence of one another;
(e) isolating the nucleic acid ligands bound to the second pool of target molecules;
(f) amplifying the isolated nucleic acid ligands bound to the second pool of target molecules;
(g) reiterating steps (a) through (f) using the amplified nucleic acid ligands as the pool of candidate nucleic acid ligands, wherein the steps are reiterated until a final pool of nucleic acid ligands is obtained with a desired level of binding specificity to the second pool of target molecules; and
(h) isolating the final pool of nucleic acid ligands so produced, wherein the final pool of nucleic acid ligands allows the differentiation of a test pool of molecules from a control pool of molecules.

The present invention further provides a method for isolating a plurality of individual nucleic acid ligands capable of binding to a plurality of different target molecules in a complex mixture of molecules, the method including the steps of:
(a) providing a pool of candidate nucleic acid ligands;
(b) providing a pool of target molecules, wherein the target molecules in the pool may be isolated;
(c) allowing the nucleic acid ligands to bind to the target molecules;
(d) isolating the nucleic acid ligands bound to the pool of target molecules;
(e) amplifying the isolated nucleic acid ligands;
(f) isolating an individual nucleic acid ligand from the amplified nucleic acid ligands;
(g) using the individual nucleic acid ligand to deplete the pool of target molecules of a specific molecule;
(h) reiterating steps (a) to (g) using the successively depleted pool of target molecules as the starting pool of target molecules for each cycle of reiteration, wherein the steps are reiterated until a plurality of individual nucleic acid ligands is identified.

It has been determined by the applicant that a nucleic acid ligand may be isolated that has the capacity to bind to a target molecule when the target molecule is present in a complex mixture of other molecules. Rather than isolating a nucleic acid ligand that has the capacity to bind to a purified or semi purified target molecule and then testing whether the nucleic acid so isolated has the capacity to bind to the target molecule when the target molecule is present in a complex mixture, it has been determined that the isolation of nucleic acid ligands that have the capacity to bind to a target molecule in a complex mixture may be achieved directly by allowing a pool of candidate single stranded nucleic acids to bind to the complex mixture itself.

This ability to isolate nucleic acid ligands to target molecules in a complex mixture may be utilised to isolate a pool of nucleic acid ligands that allows the differentiation of a test pool of molecules from a control pool of molecules. In this regard it has been further determined that the ability to isolate a pool of nucleic acid ligands capable of the differentiation of a test pool of molecules from a control pool of molecules may be achieved by a reiterative process of binding and amplification of the nucleic acid ligands to a pool of target molecules, provided that the reiterated steps of binding are performed in the presence of another pool of molecules that differs in the concentration of one or more target molecules. Without being bound by theory, it appears that any small differences in the concentration of molecules between a test pool of molecules and a control pool of molecules are magnified by the reiterated cycles of binding and amplification, and after sufficient reiterations, resulting in a final population of nucleic acids that is able to distinguish between a test pool of molecules and a control pool of molecules.

The ability to isolate nucleic acid ligands to target molecules in a complex mixture may also be utilised to isolate a plurality of individual nucleic acid ligands capable of binding to a plurality of specific target molecules in a complex mixture of molecules, by a reiterative process of binding a pool of nucleic acid ligands to a pool of target molecules, isolating the bound nucleic acid ligands, selecting an individual nucleic acid ligand, and using this nucleic acid ligand to deplete the complex mixture of the target molecule. In this way it is possible to readily isolate a plurality of nucleic acid ligands to a large number of target molecules in a complex mixture.

Various terms that will be used throughout this specification have meanings that will be well understood by a skilled addressee. However, for ease of reference, some of these terms will now be defined.

The term “nucleic acid ligand” as used throughout the specification is to be understood to mean any single stranded deoxyribonucleic acid or ribonucleic acid that may act as a ligand for a target molecule. The term includes any nucleic acid in which a modification to the sugar-phosphate backbone or a modification to the structure of the bases has been made so as to improve the capacity of the nucleic acids to act as ligands, or any other step that improves the ability to isolate, amplify or otherwise use the ligands.

The term “target molecule” as used throughout the specification is to be understood to mean any target molecule to which a nucleic acid ligand may bind. For example, target molecules may include proteins, polysaccharides, glycoproteins, hormones, receptors, lipids, small molecules, drugs, metabolites, cofactors, transition state analogues and toxins, or any nucleic acid that is not complementary to its cognate nucleic acid ligand.

The term “pool” as used throughout the specification is to be understood to mean a collection of two or more different molecules.

The term “complex mixture” as used throughout the specification is to be understood to mean a collection of two or more different target molecules. The term includes any collection of different target molecules that may be derived from a biological or non-biological source.

Examples of a complex mixture derived from a biological source include proteins, nucleic acids, oligosaccharides, lipids, small molecules (or any combination of these molecules) derived from the following sources: a cell or any part thereof, groups of cells, viral particles (or any part thereof), tissue or organ. Examples of a complex mixture from a non-biological source include complex mixtures resulting from chemical reactions.

The term “isolated” as used throughout the specification is to be understood to mean any process that results in substantial purification, in that the isolation process provides an enrichment of the species being isolated.

The term “first pool of target molecules” as used throughout the specification is to be understood to mean a first population of two or more different target molecules.

The term “control pool of molecules” as used throughout the specification is to be understood to mean a population of molecules that provides a reference population of molecules against which a change in another population is to be measured. The first pool of target molecules may be identical or similar to a control pool of molecules.

The term “second pool of target molecules” as used throughout the specification is to be understood to mean a second population of two or more different target molecules, the second population having one or more target molecules present at higher concentration than present in a first population of molecules.

The term “test pool of molecules” as used throughout the specification is to be understood to mean a population of molecules in which a change in the concentration of one or more molecular species is to be measured. The second pool of target molecules may be identical or similar to a test pool of molecules.

The term “deplete” as used throughout the specification is to be understood to mean a process by which the concentration of a specific target in a complex mixture of molecules is reduced to an extent that the concentration of the specific molecule will not provide a substantial target for the binding of nucleic acid ligands.

### BRIEF DESCRIPTION OF THE FIGURES

FIG. 1 shows labelling of a epitheliod mesothelioma with aptamer MTA R72. A bright field image of an epithelioid mesothelioma is shown in top panel and a dark field image showing staining with aptamer MTA R72 is shown in the lower panel. Predominantly nuclear staining is seen with the aptamer MTA R72. Apart from the obvious surface tumour, scattered invasive cells can also be seen in the underlying stroma.

FIG. 2 shows labeling of a a biphasic mesothelioma in which the predominant epitheliod cells are positive as well as a few spindle shaped cells. The bright field image is shown in the top panel. The pattern of binding of aptamer MTA R72 is shown in the dark field image in the bottom panel. Both the spindle and the epithelioid malignant mesothelial cells show nuclear staining.

FIG. 3 shows labeling of a desmoplastic mesothelioma with aptamer MTA R72. The desmoplastic mesothelioma demonstrates labeling of the malignant spindle cells whilst the surrounding stroma is negative (see low power shots, upper panel). The labeling appears to be cytoplasmic rather than nuclear.

FIG. 4 shows an example of binding of aptamer MTA R72 to two cases of reactive mesotheliosis, in which only very focal and weak staining is observed in reactive mesothelial cells. There is almost a complete absence of labeling apart from a ‘random’ dot-like labeling, which is quite distinct from the densely punctate staining observed in the malignant cases.

FIG. 5 shows IHC staining of mesothelioma cells with Calretinin (top left panel), negative control IHC staining of mesothelioma cells (top right panel), Cytokeratin 5/6 staining of mesothelioma cells (bottom left panel) and CD45, LCA IHC staining of mesothelioma cells (bottom right panel).

FIG. 6 shows aptamer MTA R72 staining of mesothelioma cells (top left and bottom left panels) and staining of mesolthelioma cells with a negative control (top right and bottom right panels).

FIG. 7 shows the results of binding of aptamer MTA R72 to bowel carcimona cells. The colonic adenocarcinoma demonstrates dense punctuate labelling of the invasive glands whilst the benign glands and crypts only show focal “dot-like” staining.

FIG. 8 shows the results of the binding of aptamer MTA R72 to prostate cancer cells. Cancerous cells are indicated in the tissue section (left panel) and labelling with the aptamer is shown in the right panel.

FIG. 9 shows binding of various aptamers to adenoma tissue sections.

### GENERAL DESCRIPTION OF THE INVENTION

As mentioned above, in one form the present invention provides a method for isolating a nucleic acid ligand capable of binding to a target molecule in a complex mixture, the method including the steps of:
(a) providing a pool of candidate nucleic acid ligands;
(b) providing a pool of target molecules;
(c) allowing the nucleic acid ligands to bind to the target molecules;
(d) isolating nucleic acid ligands bound to the target molecules;
(e) amplifying the isolated nucleic acid ligands;
(f) reiterating steps (a) to (e) using the amplified nucleic acid ligands as the pool of candidate nucleic acid ligands, wherein the steps are reiterated until a final pool of nucleic acid ligands is obtained with a desired level of binding specificity to the pool of target molecules; and
(g) isolating a specific nucleic acid ligand from the final pool of nucleic acid ligands, wherein the specific nucleic acid ligand is capable of binding to a target molecule in a complex mixture.

The ability to isolate a nucleic acid ligand capable of binding to a target molecule in a complex mixture allows the use of such ligands to detect and determine the concentration of target molecules in a complex mixture of molecules. The benefits of a nucleic acid ligand with such properties for diagnostic, research and treatment purposes are readily apparent. For example, such nucleic acids ligands may be used for the identification of whether a group of cells has acquired a new phenotype, such as a cancerous or pre-cancerous phenotype, by using the nucleic acid ligands to determine the concentration of important target molecule in the cells.

In addition, nucleic acids with the capacity to bind to target molecules in a complex mixture are more likely to have possible therapeutic applications, because of their ability to bind to their target in amongst a myriad of other potential targets in a complex mixture.

The nucleic acid ligands according to the methods of the present invention may be based on either deoxyribonucleic acids or ribonucleic acids. The nucleic acid ligands may also contain modifications to the sugar-phosphate backbone, modifications to the 5′ and/or 3′ ends, modifications to the 2′ hydroxyl group, the use of non-naturally occurring bases, or the use of modified bases derived from naturally or non-naturally occurring bases.

The nucleic acids according to the methods of the present invention may also be circular nucleic acid ligands or any other type of nucleic acid ligand that is conformationally restrained by intra molecular linkages.

The size of the nucleic acid ligands may be selected with regard to a number of parameters, including the desired complexity of the candidate pool and any structural and/or sequence constraints. Preferably, the pool of candidate nucleic acid ligands has an average size in the range from 30 to 150 nucleotides. More preferably, the average size is in the range from 50 to 100 nucleotides. Most preferably, the average size is 85 nucleotides.

The pool of candidate nucleic acid ligands may be generated by a method well known in the art, so long as the candidate pool generated is of sufficient complexity to allow the isolation of one or more nucleic acid ligands with the desired properties. Preferably, the pool of candidate nucleic acid ligands is generated by a method including the step of chemical synthesis. More preferably, the pool of candidate nucleic acid ligands will be generated by a method including chemical synthesis allowing the incorporation of one or more random nucleotides at a desired number of positions in the final oligonucleotides that result from the synthesis.

Preferably, the randomised section has a size in the range from 10 to 100 bases. More preferably, the randomised section has a size in the range from 30 to 80 bases. Most preferably, the randomised section is 45 bases in length.

Preferably, each of the nucleic acid ligands in the pool of candidate nucleic acid ligands includes a constant section of base sequence to allow amplification by polymerase chain reaction or to facilitate cloning.

The candidate pool may also be a pool of previously selected nucleic acid ligands. The candidate pool may also be a chemically synthesized pool of single stranded nucleic acids that has been further mutagenised by a method well known in the art or a previously selected pool of nucleic acid ligands that has been further mutagenised by a method well known in the art.

Target molecules may include proteins, polysaccharides, glycoproteins, hormones, receptors, lipids, small molecules, drugs, metabolites, cofactors, transition state analogues and toxins, or any nucleic acid that is not complementary to its cognate nucleic acid ligand.

The source of the pools of target molecules according includes cellular extracts derived from cell populations, group of cells, tissues or organs; whole cells; viral particles (or parts thereof); or chemical mixtures. Cellular extracts include extracts derived from tissues, including tissue sections and formalin fixed tissue sections. Preferably, the source of the pool of target molecules is a cellular extract. More preferably, the cellular extract is derived from human cells. Cellular extracts may be prepared by methods well known in the art.

Preferably, the cellular extract is derived from cells selected from one or more of the following types of tissue: colorectal tissue, breast tissue, cervical tissue, uterine tissue, renal tissue, pancreatic tissue, esophageal tissue, stomach tissue, lung tissue, brain tissue, liver tissue, bladder tissue, bone tissue, prostate tissue, skin tissue, ovary tissue, testicular tissue, muscle tissue or vascular tissue. These tissues may further contain cells that are normal (non-cancerous), pre-cancerous (having acquired some but not all of the cellular mutations required for a cancerous genotype) or cancerous cells (malignant or benign). Such tissues may contain cells that are normal, pre-cancerous or cancerous, any combination of cells that are normal, pre-cancerous or cancerous, or any other form of diseased cell.

As will be readily appreciated, there are numerous methods well known in the art for determining whether cells are normal, pre-cancerous, cancerous or diseased, including histopathology and other phenotypic and genotypic methods of identifying cells.

The binding of the nucleic acid ligands to the pool of target molecules of the methods of the present invention may be performed under suitable conditions known in the art. For example, the concentrations of both ligand and target, buffer composition and temperature may be selected according to the specific parameters of the particular binding reaction.

Preferably, the concentration of the nucleic acid ligands is in the range of 5 ug/ml to 50 ug/ml. As will be appreciated the concentration of the pool of target molecules will depend on the particular details of the types of target and the constituent target molecules. Preferably, the concentration of the pool of target molecules is less than or equal to 20 mg/ml.

Preferably, the binding buffer includes a phosphate buffer and/or a Tris buffer. More preferably, the binding buffer includes 10 mM phosphate. The binding buffer may also include one or more salts to facilitate appropriate binding, including NaCl and/or MgCl₂. Preferably, the binding buffer contains 0.15 M NaCl and 5 mM MgCl₂. The temperature of binding may be selected with regard to the particular binding reaction. Preferably, the binding reaction is performed at a temperature in the range from 4° C. to 40° C. More preferably, the binding reaction is performed at a temperature in the range of 20° C. to 37° C.

The isolation of the nucleic acid ligands that bind to the pool of target molecules may be achieved by a suitable method that allows for unbound nucleic acid molecules to be separated from bound nucleic acids. For example, the pool of target molecules may be functionally coupled to a solid support and unbound nucleic acid molecules removed by washing the solid support under suitable conditions.

In the case where the pool of molecules is a pool of molecules isolated from a cell extract or a biological mixture of components, such as serum, the constituent proteins may be immobilised on an activated solid support. For the immobilisation of cell extracts, activated sepharose beads are preferred for the immobilisation of proteins. Alternatively protein mixtures may be biotinylated, preferably by reacting a biotin moiety with the free amino groups of lysine residues, and using streptavidin coupled to a solid support to capture the proteins.

The washing of nucleic acids not bound to the target pool of molecules may be performed in a suitable buffer under suitable conditions well known in the art, the washing being performed until a desired level of nucleic acid ligands remaining bound to target molecules is achieved. Preferably, unbound nucleic acids are removed from the pool of target molecules by washing multiple times in the buffer used for binding.

The bound nucleic acids may then be isolated from the pool of target molecules by a suitable method well known in the art, including the washing of the bound nucleic acid ligands by a buffer of sufficient stringency to remove the bound nucleic acids. Alternatively, for nucleic acid ligands bound to cellular extracts, bound nucleic acids may be isolated by extracting both the nucleic acid ligands and the nucleic acids of the cellular extract. For example, for nucleic acid ligands bound to cellular extracts, the nucleic acids may be isolated by guanidine thiocyanate extraction, followed by acid phenol treatment and ethanol precipitation. If the nucleic acid ligand is a ribonucleic acid, the nucleic acid may first be converted to a cDNA copy by reverse transcriptase. Alternatively, for tissue extracts such as formalin-fixed tissue extracts, the tissue extract may be digested with a proteinase (for example proteinase K) in the presence of a detergent (for example sodium dodecyl sulphate) and bound nucleic acid ligands isolated in this manner.

Amplification of the isolated (ie bound) nucleic acid ligands according to the methods of the present invention may be performed by a reiterative nucleic acid amplification process well known in the art. Examples of such reiterative amplification processes include polymerase chain reaction (PCR) using appropriately designed primers, rolling circle replication and/or cloning of the nucleic acid ligands into amplifiable vectors. In the case of PCR, both symmetric and asymmetric PCR may be used. For rolling circle replication, amplification using this method may occur from circularised nucleic acid ligands as templates, or alternatively, the pool of nucleic acid ligands may be cloned (after conversion to a double stranded intermediate by synthesis of the complementary strand) into a vector and rolling circle replication performed on double or single stranded template.

The reiteration of the steps of binding and isolation of nucleic acid ligands may be performed for any number of cycles required to achieve a desired level of binding specificity of one or more of the nucleic acid ligands to the pool of target molecules. The desired level of binding specificity may be determined by a method well known in the art, including determination of the proportion of nucleic acids bound to the target molecules using detectably labelled nucleic acid ligands.

As will be appreciated, one or more individual nucleic acid ligands may then be isolated from the final pool of nucleic acid ligands. The isolation of individual nucleic acid ligands may be achieved by a method well known in art, including the cloning of the pool of nucleic acid ligands into a suitable vector and the isolation of specific clones. The cloning of the final pool may or may not include a prior step of amplification to increase the number of targets for cloning. The DNA sequence of each cloned DNA, and therefore the sequence of the nucleic acid ligand, may be determined by standard procedures if so desired.

The specific nucleic acid ligand may then be regenerated by a process including PCR, excision of DNA from the cloning vector or in vitro transcription. In the case of methods of regenerating the nucleic acid ligand that involve a double stranded nucleic acid intermediate (ie PCR and cloning), the single stranded nucleic acid may be separated from its complementary nucleic acid by a method well known in the art, including denaturing electrophoresis, denaturing HPLC or labelling of one of the strands with a moiety (for example biotin) that allows separation of the strands by electrophoresis or HPLC.

The present invention also provides a method for isolating a pool of nucleic acid ligands capable of binding to one or more target molecules in a complex mixture, the method including the steps of:
(a) providing a pool of candidate nucleic acid ligands;
(b) providing a first pool of target molecules;
(c) providing a second pool of target molecules, wherein the second pool of target molecules may be isolated from the first pool of target molecules, and wherein the second pool of target molecules differs from the first pool of target molecules in that one or more of the target molecules in the second pool is present at a higher concentration than that present in the first pool of target molecules;
(d) allowing the nucleic acid ligands to bind to the first and second pools of target molecules, wherein the first and second pool of target molecules are in the presence of one another;
(e) isolating the nucleic acid ligands bound to the second pool of target molecules;
(f) amplifying the isolated nucleic acid ligands bound to the second pool of target molecules;
(g) reiterating steps (a) through (f) using the amplified nucleic acid ligands as the pool of candidate nucleic acid ligands, wherein the steps are reiterated until a final pool of nucleic acid ligands is obtained with a desired level of binding specificity to the second pool of target molecules; and
(h) isolating the final pool of nucleic acid ligands so produced, wherein the final pool of nucleic acid ligands allows the differentiation of a test pool of molecules from a control pool of molecules.

In this form, the present invention also provides a method for isolating a pool of nucleic acid ligands capable of binding to one or more target molecules in a complex mixture, wherein the pool of nucleic acid ligands allows the differentiation of a test pool from a control pool of molecules.

Preferably, the first pool of target molecules and the second pool of target molecules are both derived from cellular extracts. As such, the cellular extracts may include nucleic acids, proteins, oligosaccharides, small molecules and lipids. Preferably, the second pool of target molecules is derived from a population of cells phenotypically or genotypically similar to the population of cells from which the first pool of target molecules is derived.

The first pool of target molecules is preferably a cellular extract, including a cellular extract derived from a tissue, including tissue sections and formalin fixed tissue sections. More preferably, the cellular extract is derived from human cells. Cellular extracts may be prepared by methods well known in the art.

Preferably, the first pool of target molecules is a cellular extract derived from cells selected from one or more of the following types of tissue: colorectal tissue, breast tissue, cervical tissue, uterine tissue, renal tissue, pancreatic tissue, esophageal tissue, stomach tissue, lung tissue, brain tissue, liver tissue, bladder tissue, bone tissue, prostate tissue, skin tissue, ovary tissue, testicular tissue, muscle tissue or vascular tissue. These tissues may contain cells that are normal (non-cancerous), pre-cancerous (having acquired some but not all of the cellular mutations required for a cancerous genotype) or cancerous cells (malignant or benign). Such tissues may contain cells that are normal, pre-cancerous or cancerous, any combination of cells that are normal, pre-cancerous or cancerous, or any other form of diseased cell.

More preferably, the first pool of target molecules is a cellular extract derived from normal or pre-cancerous cells.

The second pool of target molecules is preferably a cellular extract, including a cellular extract derived from a tissue, including tissue sections and formalin fixed tissue sections. More preferably, the cellular extract is derived from human cells.

Preferably, the second pool of target molecules is a cellular extract derived from cells selected from one or more of the following types of tissue: colorectal tissue, breast tissue, cervical tissue, uterine tissue, renal tissue, pancreatic tissue, esophageal tissue, stomach tissue, lung tissue, brain tissue, liver tissue, bladder tissue, bone tissue, prostate tissue, skin tissue, ovary tissue, testicular tissue, muscle tissue or vascular tissue These tissues may contain cells that are normal (non-cancerous), pre-cancerous (having acquired some but not all of the cellular mutations required for a cancerous genotype) or cancerous cells (malignant or benign). Such tissues may contain cells that are normal, pre-cancerous or cancerous, any combination of cells that are normal, pre-cancerous or cancerous, or any other form of diseased cells.

More preferably, the second pool of target molecules is a cellular extract derived from pre-cancerous or cancerous cells.

The binding of the nucleic acid ligands to the first pool of target molecules in the presence of a second pool of target molecules may be performed under suitable conditions and in a suitable buffer. Preferably, the first pool of molecules will be in a molar excess to the second pool of molecules for the binding of the nucleic ligands. More preferably, the first pool of molecules will be in a ten fold or greater molar excess to the second pool of molecules for the binding of the nucleic ligands.

This form of the present invention requires the ability of the nucleic acid ligands binding to the second pool of target molecules to be isolated from the first pool of target molecules. The isolation of the second pool of target molecules from the first pool of target molecules may be achieved by the spatial separation of the pools of targets on a solid support, so that the isolation of the second pool of molecules may be achieved by isolating that part of the solid support containing the second pool of target molecules. For example, in the case whereby fixed tissue sections containing normal cells and a group of abnormal cells are used, the abnormal fixed cells will be physically separated from the normal fixed cells. Isolation of the second pool of target molecule with bound nucleic acid ligands may be accomplished by physically removing the portion of solid support having the second pool of target molecules bound to it.

Alternatively, the isolation of the second pool of target molecules from the first pool of nucleic acids may be achieved by a method that allows the separation of the first pool of target molecules from the second pool. For example, a first pool of normal cells may be isolated from a second pool of diseased cells by a method such as FACS (fluorescence activated cell sorting) or the capture of cells by antibodies to specific cell surface antigens. Alternatively, the different cells may be isolated by using a specific molecule that binds to a cell surface marker and which is attached to a solid support, such as a magnetic bead. Also, chemical coupling techniques may be used to couple a selectable moiety to the second pool of target molecules, and thereby allow isolation of the second pool of molecules from the first pool of target molecules. A further method of isolating cells is the use of laser capture microscopy.

The washing of the nucleic acids to remove nucleic acids not bound to the second pool of molecules may be achieved using a suitable buffer under suitable conditions. For the washing of nucleic acids bound to cellular extracts, the first pool of target molecules and the second pool of target molecules with bound nucleic acid ligands may or may not be washed together. Preferably, the washing involves washing multiple times in the original binding buffer as a means to remove unbound nucleic acid ligands.

The reiteration steps of this form of the present invention are continued until the desired level of binding specificity to the second pool of target molecules is achieved. Preferably the reiterations are continued until the proportion of the nucleic binding to the second pool of target molecules does not show any significant increase. The determination of the proportion of nucleic acid ligands binding to the second pool may be achieved by a method well known in the art, including detectably labelling a proportion of the nucleic acid ligands and determining the extent of binding. Detection of the nucleic acids ligands by a biotin:steptavidin method is preferred.

Alternatively, the steps may be reiterated until the pool of nucleic acid ligands shows specific binding to the target cell population and exhibits only a lower or background binding to other regions. Detection of the nucleic acids ligands by a biotin:steptavidin method is preferred.

The final pool of nucleic acid ligands so produced will allow the differentiation of a test pool of molecules from a control pool of molecules. The differentiation may be achieved by methods well known in the art including detectably labelling the final pool of nucleic acid ligands and determining the extent of binding to the test pool of molecules and the control pool of molecules. Detection of the nucleic acids ligands by a biotin:steptavidin method is preferred.

The test pool of target molecules is preferably a cellular extract, including a cellular extract derived from a tissue, including tissue sections and formalin fixed tissue sections. More preferably, the cellular extract is derived from human cells.

Preferably, the test pool of target molecules is a cellular extract derived from cells selected from one or more of the following types of tissue: colorectal tissue, breast tissue, cervical tissue, uterine tissue, renal tissue, pancreatic tissue, esophageal tissue, stomach tissue, lung tissue, brain tissue, liver tissue, bladder tissue, bone tissue, prostate tissue, skin tissue, ovary tissue, testicular tissue, muscle tissue or vascular tissue. These tissues may contain cells that are normal (non-cancerous), pre-cancerous (having acquired some but not all of the cellular mutations required for a cancerous genotype) or cancerous cells (malignant or benign). Such tissues may contain cells that are normal, pre-cancerous or cancerous, any combination of cells that are normal, pre-cancerous or cancerous, or any other form of diseased cells.

More preferably, the test pool of target molecules is a cellular extract derived from pre-cancerous or cancerous cells. Most preferably, the test pool of molecules is a cellular extract derived from cells that are the same, or genotypically or phenotypically similar, to the cells from which the cellular extract of the second pool of target molecules is derived.

The control pool of target molecules is preferably a cellular extract, including a cellular extract derived from a tissue, including tissue sections and formalin fixed tissue sections. More preferably, the cellular extract is derived from human cells.

Preferably, the control pool of target molecules is a cellular extract derived from cells selected from one or more of the following types of tissue: colorectal tissue, breast tissue, cervical tissue, uterine tissue, renal tissue, pancreatic tissue, esophageal tissue, stomach tissue, lung tissue, brain tissue, liver tissue, bladder tissue, bone tissue, prostate tissue, skin tissue, ovary tissue and testicular tissue. These tissues may contain cells that are normal (non-cancerous), pre-cancerous (having acquired some but not all of the cellular mutations required for a cancerous genotype) or cancerous cells (malignant or benign). Such tissues may contain cells that are normal, pre-cancerous or cancerous, any combination of cells that are normal, pre-cancerous or cancerous, or any other form of diseased cells.

More preferably, the control pool of target molecules is a cellular extract derived from normal or pre-cancerous cells. Most preferably, the control pool of molecules is a cellular extract derived from cells that are the same, or genotypically or phenotypically similar, to the cells from which the cellular extract of the first pool of target molecules is derived.

This form of the present invention also contemplates the isolation of one or more individual nucleic acid ligands from the final pool, each of the nucleic acid ligands so isolated being capable of binding to a target molecule in a complex mixture, such as the complex mixture present in the second pool of target molecules or the complex mixture in the test pool of molecules.

Accordingly, in a preferred form the present invention also provides a method for isolating a nucleic acid ligand capable of binding to a target molecule in a complex mixture, the method including the steps of:
(a) providing a pool of candidate nucleic acid ligands;
(b) providing a first pool of target molecules;
(c) providing a second pool of target molecules, wherein the second pool of target molecules may be isolated from the first pool of target molecules, and wherein the second pool of target molecules differs from the first pool of target molecules in that one or more of the target molecules present in the second pool is present at a higher concentration than that present in the first pool of target molecules;
(d) allowing the nucleic acid ligands to bind to the first and second pools of target molecules, wherein the first and second pool of target molecules are in the presence of one another;
(e) isolating the nucleic acid ligands bound to the second pool of target molecules;
(f) amplifying the isolated nucleic acid ligands bound to the second pool of target molecules;
(g) reiterating steps (a) through (f) using the amplified nucleic acid ligands as the pool of candidate nucleic acid ligands, wherein the steps are reiterated until a final pool of nucleic acid ligands is obtained with a desired level of binding specificity to the second pool of target molecules;
(h) isolating the final pool of nucleic acid ligands so produced, wherein the final pool of nucleic acid ligands allows the differentiation of a test pool of molecules from a control pool of molecules; and
(i) isolating a nucleic acid ligand from the final pool of nucleic acid ligands, wherein the isolated nucleic acid ligand is capable of binding to a target molecule in a complex mixture.

Preferably, the nucleic acid ligand isolated (which is capable of binding to a target molecule in the complex mixture) allows the differentiation of a test pool of molecules from a control pool of molecules.

The present invention further provides a method for isolating a plurality of individual nucleic acid ligands capable of binding to a plurality of different target molecules in a complex mixture of molecules, the method including the steps of:
(a) providing a pool of candidate nucleic acid ligands;
(b) providing a pool of target molecules, wherein the target molecules in the pool may be isolated;
(c) allowing the nucleic acid ligands to bind to the target molecules;
(d) isolating the nucleic acid ligands bound to the pool of target molecules;
(e) amplifying the isolated nucleic acid ligands;
(f) isolating an individual nucleic acid ligand from the amplified nucleic acid ligands;
(g) using the individual nucleic acid ligand to deplete the pool of target molecules of a specific molecule;
(h) reiterating steps (a) to (g) using the successively depleted pool of target molecules as the starting pool of target molecules for each cycle of reiteration, wherein the steps are reiterated until a plurality of individual nucleic acid ligands is identified.

In this form the present invention provides a method for the isolation of a plurality of individual nucleic acids capable of binding to a plurality of specific molecules in a complex mixture of molecules. The ability to isolate a plurality of individual nucleics may be useful, for example, for monitoring the extent of expression of a number of molecules simultaneously in a complex mixture.

As will be appreciated, in this form a nucleic acid ligand is isolated from a pool of nucleic acid ligands that binds to a complex mixture and the nucleic acid ligand so isolated is then used to deplete the complex mixture of the specific target molecule that binds the ligand. The process is then reiterated until a plurality of nucleic acid ligands capable of binding to a plurality of specific molecules is achieved. Accordingly, the present invention further contemplates one or more individual nucleic acid ligands isolated from the plurality of nucleic acid ligands isolated by this method.

To deplete the pool of target molecules, an individual nucleic acid ligand may be produced in large quantities and coupled to a solid support. Chemical synthesis methods (if the nucleotide sequence of the ligand has been determined), PCR amplification or in vitro transcription (for RNA nucleic acid ligands) are preferred methods for producing quantities of the nucleic acid ligand suitable for coupling to the solid support.

The depletion of the specific molecule from the pool of target molecules may be achieved by passing the pool of target molecules over the nucleic acid ligand bound to the solid support and retaining the eluate. For example, biotinylated oligonucleotides may be used as the nucleic acid ligand, and the depletion of the specific molecule from the pool of target molecules may be achieved by allowing the specific molecule to bind to an excess of the oligonucleotide, and then isolating the nucleic acid-protein complex by binding the oligonucleotide to streptavidin paramagentic beads.

The remaining eluate is then to be used in the next round of binding as the pool of target molecules. In this manner the eluate becomes successively depleted in specific molecules, and specifically enriched for those molecules to which a nucleic acid ligand has not been identified.

The process may then be reiterated to isolate new nucleic acid ligands to one or more of the remaining targets molecules in the depleted pool of targets using a fresh candidate pool of nucleic acid ligands for each round. Alternatively, the pool of nucleic acid ligands that bound to the pool of target molecules may be used as the candidate pool of nucleic acid ligands. In this case, it may be necessary to further amplify this pool of nucleic acid ligands so as to attain a concentration of nucleic acid ligands that may be used as the starting pool of candidate nucleic acid ligands.

As will be appreciated, multiple nucleic acid ligands may also be used at each cycle of reiteration to accelerate the identification of nucleic acid ligands.

Reiteration of the process allows the isolation of a plurality of individual nucleic acid ligands capable of binding to a plurality of specific molecules in a complex mixture of molecules. Eventually, such a process should yield a nucleic acid ligand for every molecule in a complex pool of targets.

The identification of a plurality of individual nucleic acid ligands capable of binding to a plurality of specific molecules in a complex mixture of molecules may then be used to determine the individual concentration of each specific molecule so identified in the complex.

Preferably, the plurality of individual nucleic acid ligands can be used to determine the concentration of a plurality of specific molecules in a target complex by using each individual nucleic acid as a separate ligand in a quantifiable system. For example, the quantifiable system may consist of a system in which the individual nucleic acid ligand is coupled to a solid support and the concentration of the specific molecule is determined by surface plasmon resonance or fluorescence correlation spectroscopy. Diagnostic applications of the method of the present invention may then be envisaged.

As will be appreciated, the identity of the specific molecule to which the isolated individual nucleic acid ligands binds may also be determined if so desired. This may be achieved by methods well known in the art, including coupling a suitable amount of the single stranded DNA to a solid support and purifying the target molecule by affinity chromatography. Preferably, microspheres or nanospheres are preferred for the coupling of the isolated individual nucleic acid ligand to a solid support. Once the target molecule has been substantially purified, the identity of the molecule may be determined by a suitable means. Mass spectrometry methods for determining the identity of the specific molecule are preferred.

The present invention also provides a polynucleotide including the nucleotide sequence according to SEQ ID No. 1.

In this form of the present invention, a polynucleotide with the following sequence is provided:

| | |
|---|---|
| 5′GGGAGCTCAGAATAAACGCTCAAGGAACAGCAAGATA | SEQ ID NO:1 |
| CGGTCACCGAACATAGCGCACCACAGGCACA3′.: | |

This nucleotide sequences is the sequence of a nucleic acid ligand capable of distinguishing malignant mesothelioma cells from non malignant mesothelial cells. This nucleic acid ligand is also capable of distinguishing malignant and non-malignant lung cells, malignant and non-malignant cells of the bowel, and malignant and non-malignant prostate cancer cells.

The polynucleotide according to the various forms of the present invention may be modified at one or more base moieties, sugar moieties, or the phosphate backbone, and may also include other appending groups to facilitate the function of the polynucleotide to function as a nucleic acid ligand or as a diagnostic reagent.

For example, the polynucleotide may include at least one modified base moiety, such as 5-fluorouracil, 5-bromouracil, 5-chlorouracil, 5-iodouracil, hypoxanthine, xanthine, 4-acetylcytosine, 5-(carboxyliydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil5-oxyacetic acid methylester, uracil-5-oxyacetic acid (v), 5-methyl-2-thiouracil, 3-(3-amino-3-N-2-carboxypropyl) uracil, (acp3) w, and 2,6-diaminopurine.

The polynucleotide may also include at least one modified sugar moiety such as arabinose, 2-fluoroarabinose, xylulose, and hexose.

The polynucleotide according to the various forms of the present invention may be synthesized, purified and isolated by a method known in the art. For example, phosphorothioate polynucleotides may be synthesized by the method as described in Stein et al. (1988) Nucl. Acids Res. 16: 3209. Alternatively, the polynucleotide may be synthesized as a double stranded DNA by an amplification reaction such as PCR from a DNA template, and the complementary strand removed by either isolating the single strand with the polynucleotide or by digesting the complementary strand (phosphorylated at its 5′ end) with an enzyme such as lambda exonuclease.

The polynucleotide according to the present invention may consist only of the nucleotide sequence of SEQ ID NO:1, or alternatively, may further include one or more flanking nucleotide sequences. For example, the polynucleotide may include one or more flanking sequences that are used to amplify the polynucleotide sequence, and/or 5′ and 3′ capping structures known in the art to provide further stability to the polynucleotide in vitro or in vivo.

The polynucleotide of the present invention is useful as diagnostic reagent for identifying at least one difference at the molecular level between malignant and non-malignant cells. In particular, the polynucleotide is capable of identifying at least one difference at the molecular level between the following malignant and non-malignant cell types:
(i) malignant mesothelioma cells (including epithelioid mesothelioma cells, biphasic mesothelioma cells, desmoplastic mesothelioma cells and sarcomatoid mesothelioma cells) and non-malignant mesothelial cells (including benign or reactive mesothelial cells);
(ii) malignant lung cells (including lung adenocarcinoma cells, lung small cell carcinoma cells, lung large carcinoma cells and lung squamous cell carcinoma cells) and non-malignant lung cells;
(iii) malignant bowel cells (bowel adenoma cells and bowel carcinoma cells) and non-malignant bowel cells; and
(iv) malignant prostate cells and non-malignant prostate cells.

The polynucleotide of the various forms of the present invention may be routinely adapted for diagnostic purposes as a nucleic acid ligand according to any number of techniques employed by those skilled in the art. The nucleic acid ligand may be labelled by procedures known in the art in order to track the presence of the ligand. For example, the nucleic acid ligand may be labelled with biotin and the nucleic acid ligand detected by way of a biotin:streptavidin complex.

The present invention also provides a polynucleotide including a variant of the nucleotide sequence according to SEQ ID NO.1, wherein the polynucleotide forms a nucleic acid ligand that identifies at least one difference at the molecular level between two complex biological mixtures.

In this regard, the term “complex biological mixture” as used throughout the specification is to be understood to mean a collection of two or more different target molecules derived from a biological source. For example, the complex biological mixture may be a cellular extract derived from a cell (such as a cell present in a formalin fixed tissue, or an extract of molecules from one or more cells such as blood plasma). The complex biological mixture may also be an isolated cell (such as cell in tissue culture or a cell isolated from a biological source, such as a cell isolated by FACS), the complex biological mixture may be one or more cells present in a tissue sample, a biological fluid (such as blood) or in a biopsy, or the complex biological mixture one or more cells present in an entire human or animal.

In addition, the term “variant” as used throughout the specification will be understood to mean any DNA or RNA polynucleotide that is a fragment of SEQ ID NO:1, or any DNA or RNA polynucleotide that contains one or more base substitutions, deletions or insertions of the nucleotide sequence of SEQ ID NO:1 or a fragment of this polynucleotides. The variant will be capable of forming a nucleic acid ligand that identifies at least one difference at the molecular level between two complex biological systems.

In this regard, it will be appreciated that the polynucleotide sequence according to SEQ ID NO:2 (aptamer MTA R720), which is capable of distinguishing malignant mesothelioma cells from non-malignant mesothelial cells, is a variant of SEQ ID NO:1.

| | |
|---|---|
| 5′GGGAGCTCAGAATAAACGCTCAACAAAAGACTATCCA | SEQ ID NO:2 |
| GCGACACGCAATCTCAAGCAACAGAGGACAG3′: | |

In the case where the variant is a fragment of SEQ ID NO:1, the fragment may be any DNA or RNA polynucleotide. A nucleotide sequence including one or more base substitutions, deletions or insertions of the nucleotide sequence according to SEQ ID NO:1 is any DNA or RNA polynucleotide that contains one or more base substitutions, deletions or insertions of the nucleotide sequence of SEQ ID NO:1, or a fragment thereof. Such variants will also be capable of forming a nucleic acid ligand that identifies at least one difference at the molecular level between two complex biological mixtures.

Preferably, the polynucleotide forms a nucleic acid ligand that identifies at least one difference at the molecular level between malignant and non-malignant cells. More preferably, the polynucleotide forms a nucleic acid ligand that identifies at least one difference at the molecular level between malignant and non-malignant cells present in a formalin fixed tissue sample.

Preferably, the polynucleotide forms a nucleic acid ligand that identifies at least one difference at the molecular level between the following malignant and non-malignant cells:
(i) malignant mesothelioma cells (including epithelioid mesothelioma cells, biphasic mesothelioma cells, desmoplastic mesothelioma cells and sarcomatoid mesothelioma cells) and normal lung cells or benign or reactive mesothelial cells;
(ii) malignant lung cells (including lung adenocarcinoma cells, lung small cell carcinoma cells, lung large carcinoma cells and lung squamous cell carcinoma cells) and non-malignant lung cells;
(iii) malignant bowel cells (bowel adenoma cells and bowel carcinoma cells) and non-malignant bowel cells; and
(iv) malignant prostate cells and non-malignant prostate cells.

The ability of the polynucleotide to form a nucleic acid ligand that identifies at least one difference at the molecular level between two complex biological mixtures may be confirmed by exposing the nucleic acid ligand under the appropriate conditions to each of the complex biological mixtures and detecting the extent of differential binding of the nucleic acid ligand to the mixtures.

For example, for distinguishing between malignant mesothelioma cells and non-malignant mesothelial cells, formalin fixed tissue sections may be used. In this case, the sections may be de-paraffinised and washed through a series of graded alcohol before undergoing antigen retrieval (121° C. in sodium citrate buffer pH 6.5 for 12 min, then left to cool for 2 hrs). The antigen retrieved tissue sections may then be equilibrated in binding buffer (1×PBS, 5 mM MgCl₂) and incubated overnight in a humidified chamber with thermally equilibrated nucleic acid ligand (1 nM). The sections may then be thoroughly washed in binding buffer to remove unbound ligand and the ligand detected. An Enzyme Labelled Fluorescence (ELF) kit (Molecular Probes, USA) is suitable for this purpose. In this instance, the biotinylated ligand is bound to streptavidin which is bound to alkaline phosphatase that reacts with the ELF substrate. This reaction produces an intensely fluorescent yellow green precipitate at the site of enzymatic activity.

A similar procedure is also suitable for distinguishing malignant lung cells (including lung adenocarcinoma cells, lung small cell carcinoma cells, lung large carcinoma cells and lung squamous cell carcinoma cells) from non-malignant lung cells, malignant bowel cells (bowel adenoma cells and bowel carcinoma cells) from non-malignant bowel cells, and malignant prostate cells from non-malignant prostate cells.

In the case of a variant which is a base substitution, insertion and/or deletion of SEQ ID NO:1, preferably the variant polynucleotide includes 5 or less base changes from the primary sequence of SEQ ID NO:1, more preferably 3 or less base changes from the primary sequence of SEQ ID NO:1, and most preferably 1 base change from the primary sequence of SEQ ID NO:1.

Preferably the variant has at least 80% sequence identity with SEQ ID NO:1, more preferably at least 90% sequence identity with SEQ ID NO:1, more preferably at least 95% sequence identity with SEQ ID NO:1, and most preferably at least 98% sequence identity with SEQ ID NO:1.

Various algorithms known in the art exist for determining the degree of homology between any two nucleic acid sequences. For example, the BLAST algorithm can be used for determining the extent of sequence homology between two sequences. BLAST identifies local alignments between two sequences and predicts the probability of the local alignment occurring by chance. The BLAST algorithm is as described in Altschul et al., 1990, J. Mol. Biol. 215:403-410.

A fragment of SEQ ID NO:1 may be synthesized, purified and isolated by a method known in the art. For example, phosphorothioate polynucleotides may be synthesized by the method as described in Stein et al. (1988) Nucl. Acids Res. 16: 3209. Alternatively, the fragment may be synthesized as a double stranded DNA by an amplification reaction such as PCR from a DNA template, and the complementary strand removed by either isolating the single strand with the polynucleotide according to SEQ ID NO:1, or by digesting the complementary strand (phosphorylated at its 5′ end) with an enzyme such as lambda exonuclease.

In the case of the variant being a base substitution, deletion or insertion, the polynucleotide may also synthesized in vitro, with the appropriate substitution, deletion or insertion being incorporated during the synthesis reaction. Alternatively, a clone having the cloned aptamer sequence may be mutagenised to incorporate a base substitution, deletion or insertion by a method known in the art.

The present invention also provides a polynucleotide sequence that hybridises with the complement of SEQ ID NO.1 under stringent hybridisation conditions, wherein the polynucleotide forms a nucleic acid ligand that identifies at least one difference at the molecular level between two complex biological mixtures.

In this regard, the term “hybridises” or “hybridisation” (or variants thereof) is to be understood to mean any process by which a strand of nucleic acid binds with a complementary strand through base pairing. Hybridisation may occur in solution, or between one nucleic acid sequence present in solution and another nucleic acid sequence immobilized on a solid support (e.g., membranes, filters, chips etc).

In addition, the term “stringent conditions” is to be understood to mean the conditions that allow complementary nucleic acids to bind to each other within a range from at or near the Tm (Tm is the melting temperature) to about 20° C. below Tm. Factors such as the length of the complementary regions, type and composition of the nucleic acids (DNA, RNA, base composition), and the concentration of the salts and other components (e.g. the presence or absence of formamide, dextran sulphate and/or polyethylene glycol) must all be considered, essentially as described in Current Protocols in Molecular Biology, John Wiley & Sons, N.Y. (1989).

An example of stringent conditions is hybridisation at 4×SSC at 65° C., followed by a washing in 0.1×SSC at 65° C. for one hour.

For example, the polynucleotide with a nucleotide sequence which is the complement of SEQ ID NO:1 may be immobilised on a filter, as described in Sambrook, J, Fritsch, E. F. and Maniatis, T. Molecular Cloning: A Laboratory Manual 2nd. ed. Cold Spring Harbor Laboratory Press, New York. (1989). To determine if the polynucleotide of interest hybridises to the complement of SEQ ID NO:1, the conditions allowing hybridisation under stringent conditions: prehybridization may be performed in a prehybridization solution (eg 4×SSC (1×=100 mM NaCl, 10 mM sodium citrate, pH 7.0), 5× Denhardt's reagent (1 g/l each of Ficoll, Polyvinyl-pyrrolidone, Bovine Serum Albumin), 1.0% SDS, 100 ug/ml denatured, fragmented salmon sperm DNA) for 2 to 12 hours. Hybridizition of the probe with the target (ie filter) may then be performed under conditions such as 4×SSC, 1.0% SDS, 100 ug/ml denatured, fragmented salmon sperm DNA, at 65° C. overnight. The filter may then be washed with 0.1×SSC and 0.1% SDS at room temperature for 15 min at 20° C.

Accordingly, in a preferred form the present invention provides a polynucleotide which hybridises with the complement of the nucleotide sequence according to SEQ ID NO.1 under stringent hybridisation conditions, wherein the polynucleotide is capable of forming a nucleic acid ligand that identifies at least one difference at the molecular level between two complex biological mixtures, and wherein the stringent hybridisation conditions include hybridisation in 4×SSC at 65° C. and washing in 0.1×SSC at 65° C.

Another example of stringent conditions is hybridisation at 42° C. in a solution including 50% formamimide, 5×SSC and 1% SDS or at 65° C. in a solution including 5×SSC and 1% SDS, with a wash in 0.2×SSC and 0.1% SDS at 65° C.

Accordingly, in another preferred form the present invention provides a polynucleotide sequence which hybridises with the complement of SEQ ID NO.1 under stringent hybridisation conditions, wherein the polynucleotide is capable of forming a nucleic acid ligand that identifies at least one difference at the molecular level between two complex biological mixtures, and wherein the stringent hybridisation conditions include hybridisation in 50% formamide, 5×SSC and 1% SDS at 65° C. and washing in 0.2×SSC and 0.1% SDS at 65° C.

As described previously, the ability of the polynucleotide to form a nucleic acid ligand that identifies at least one difference at the molecular level between two complex biological mixtures may be confirmed by exposing the nucleic acid ligand under the appropriate conditions to each of two complex biological mixtures and detecting the extent of differential binding of the nucleic acid ligand to the mixtures.

As described previously, the polynucleotide may be synthesized, purified and isolated by a method known in the art. For example, phosphorothioate polynucleotides may be synthesized by the method as described in Stein et al. (1988) Nucl. Acids Res. 16: 3209.

The present invention also provides a nucleic acid ligand that distinguishes a malignant cell from a non-malignant cell.

Preferably, the nucleic acid ligand includes a nucleotide sequence according to SEQ ID NO:1 to SEQ ID NO:32, or a nucleotide sequence which is a variant of SEQ ID NO:1 to SEQ ID NO:32.

The nucleotide sequence of the various nucleic acid ligands is as follows:

| | |
|---|---|
| MTA R72 | |
| 5′GGGAGCTCAGAATAAACGCTCAAGGAACAGCAAGATACGGTCACCGAAC | (SEQ ID NO:1) |
| ATAGCGCACCACAGGCAC3′ | |
| MTA R720 | |
| 5′GGGAGCTCAGAATAAACGCTCAACAAAAGACTATCCAGCGACACGCAAT | (SEQ ID NO:2) |
| CTCAAGCAACAGAGGACAG3′ | |
| MTA R78 | |
| 5′GGGAGCTCAGAATAAACGCTCAAGCCATGGACAAGACTAACGACAGACC | (SEQ ID NO:3) |
| TAAACCTAAAGGATAAAAA3′ | |
| MTA R73 | |
| 5′GGGAGCTCAGAATAAACGCTCAAACCCGAAAAGCGGGAAAACCCCCAG | (SEQ ID NO:4) |
| CAAATCCCGACCAAAAGCAA3′ | |
| MTA R74 | |
| 5′GGGAGCTCAGAATAAACGCTCAACCTGTTTTTTTTCCCCCTTATTCTTCC | (SEQ ID NO:5) |
| CCCCCCTGTGTCGC3′ | |
| MTA R75 | |
| 5′GGGAGCTCAGAATAAACGCTCAATTGTGTGTCTTCTTGCTCTTCTTCCTT | (SEQ ID NO:6) |
| CCCTTGCCTTGCCATTGT3′ | |
| MTA R76 | |
| 5′GGGAGCTCAGAATAAACGCTCAATGTGTGCTGTCAGGCGTGCTGTGTGT | (SEQ ID NO:7) |
| GTAATCTTGGTGCGGCCTC3′ | |
| MTA R77 | |
| 5′GGGAGCTCAGAATAAACGCTCAAGCAGGACCAAGAAGACACCCAAAAAG | (SEQ ID NO:8) |
| AAGCAATGAAAGAGGCAG3′ | |
| MTA R71 | |
| 5′GGGAGCTCAGAATAAACGCTCAAGTTGGGGTGTTGCCTGGTCTGTGTAC | (SEQ ID NO:9) |
| GCTGGGGGCGGTTGCGTGG3′ | |
| Prostate R611: | |
| 5′GGGAGCTCAGAATAAACGCTCAACAATTTTCTTTTTCCCTTCTCTGTCCT | (SEQ ID NO:10) |
| TTTCTCCGTGCTTG3′ | |
| Prostate R612: | |
| 5′GGGAGCTCAGAATAAACGCTCAACAATTTTCTTTTTCCCTTCTCTGTCCT | (SEQ ID NO:11) |
| TTTCTCCGTGCTTG3′ | |
| Prostate R630 | |
| 5′GGGAGCTCAGAATAAACGCTCAAGTTTTTCCTCCTGCCTGTTTTCTTCCC | (SEQ ID NO:12) |
| CGTGCTCCTTTTCCCCCC3′ | |
| Prostate R68 | |
| 5′GGGAGCTCAGAATAAACGCTCAAAAGAATCAGCAGAGACAGGGAGGCG | (SEQ ID NO:13) |
| AGAAAGAAGGGGGGGGGGGAG3′ | |
| Prostate R623 | |
| 5′GGGAGCTCAGAATAAACGCTCAACAGCCAGGACAGAGAGGTGGGAAAC | (SEQ ID NO:14) |
| CCAAACAAGAGCAAATAGCC3′ | |
| Prostate R812: | |
| 5′GGGAGCTCAGAATAAACGCTCAATTGTTTTGGCTTTGTCTCCCGTTTGCC | (SEQ ID NO:15) |
| TTCCCCGGCCTTTGTCTG3′ | |
| Prostate R823: | |
| 5′GGGAGCTCAGAATAAACGCTCAATCTGGGTCTGTGTGTATCTTTTCCATT | (SEQ ID NO:16) |
| GCCTCCTTCCCTTCGTCT3′ | |
| Prostate R842: | |
| 5′GGGAGCTCAGAATAAACGCTCAATCTTGCCGGTTCTCTCCTTTTTCCTGT | (SEQ ID NO:17) |
| CTGCCTTCTTTCTCCTTG3′ | |
| Prostate R1023 | |
| 5′GGGAGCTCAGAATAAACGCTCAACCTCCTGTCTGCTCCTATCTCTTGCCT | (SEQ ID NO:18) |
| TCCTTGTTTCCCCTGCC3′ | |
| Prostate R104 | |
| 5′GGGAGCTCAGAATAAACGCTCAACTCTGTCCTTTCCCTTTCTCCCTTTCT | (SEQ ID NO:19) |
| TGCTGCTCCTTTCGTGTC3′ | |
| Prostate R1046 | |
| 5′GGGAGCTCAGAATAAACGCTCAACACTTTTCTTGTCCATTTGCTTCTCTA | (SEQ ID NO:20) |
| CCCTCATTCTCCCATCCT3′ | |
| Prostate R1011 | |
| 5′GGGAGCTCAGAATAAACGCTCAAAGCCTCTCTACCGTGGTGCTGCCCTT | (SEQ ID NO:21) |
| CGATTTGTGTCTGCTGTGT3′ | |
| Prostate R1013 | |
| 5′GGGAGCTCAGAATAAACGCTCAATGTGGTTTTGCCTTTTCTTTCCGTTTT | (SEQ ID NO:22) |
| CTCTTTCCCTCGCGCCT3′ | |
| Prostate R1010 | |
| 5′GGGAGCTCAGAATAAACGCTCAACCCCCTGCTTCCCCCTCCTTATGTTG | (SEQ ID NO:23) |
| TCCTGCCGGCGCCTGTACT3′ | |
| Prostate R1020 | |
| 5′GGGAGCTCAGAATAAACGCTCAATCACTGTCGTCATTTATTTTTTCAGTC | (SEQ ID NO:24) |
| CTATTTCCCTCTCCTGTG3′ | |
| Prostate R1030 | |
| 5′GGGAGCTCAGAATAAACGCTCAATCCTCTTTTTTGTACGGCCTGCTGTTT | (SEQ ID NO:25) |
| GTCTGTGTGTCTTCCTCA3′ | |
| Prostate R1031 | |
| 5′GGGAGCTCAGAATAAACGCTCAACCAGTCGGCTCCTTTCCTGCGCGTCT | (SEQ ID NO:26) |
| CTTCCCGTTTTTTCCCCCT3′ | |
| Prostate R1044 | |
| 5′GGGAGCTCAGAATAAACGCTCAATGTTGCCTAATTCCTGCTATGTTTCTG | (SEQ ID NO:27) |
| TCTCCTCCCCACCCGCGC3′ | |
| Adenoma R832 | |
| 5′GGGAGCTCAGAATAAACGCTCAAGCCCCCATAGCAGCAAAGTAAGAACA | (SEQ ID NO:28) |
| ACCAACAGACGCACGACGG3′ | |
| Adenoma R839 | |
| 5′GGGAGCTCAGAATAAACGCTCAACCAAAAGAACACAACAGAACCAAGCA | (SEQ ID NO:29) |
| GACACCCACACCACCGCAG3′ | |
| Adenoma R846 | |
| 5′GGGAGCTCAGAATAAACGCTCAAAGTTGGTGTTTTCCTTTCCCTGTCCC | (SEQ ID NO:30) |
| CTTGTTTCATCTTCCCTAC3′ | |
| Adenoma R838 | |
| 5′GGGAGCTCAGAATAAACGCTCAATCCCTTTTTCCCATCTTTTCGCGGTTG | (SEQ ID NO:31) |
| TTGAGCTTTCTGCGTGTG3′ | |
| Adenoma R834 | |
| 5′GGGAGCTCAGAATAAACGCTCAAGGACCAGCACACACACCAACAAAGGC | (SEQ ID NO:32) |
| CAGGGACCCGGTACCCACC3′ | |

Nucleic acid ligands with the nucleotide sequence according to SEQ ID NO: 1 through SEQ ID NO:9 are useful for distinguishing malignant mesothelioma cells (including epithelioid mesothelioma cells, biphasic mesothelioma cells, desmoplastic mesothelioma cells and sarcomatoid mesothelioma cells) from non-malignant mesothelial cells or benign or reactive mesothelial cells.

As such, the present invention contemplates the following further forms of the present invention:
(i) A polynucleotide including the nucleotide sequences according to one of SEQ ID NO:1 to SEQ ID NO:9;
(ii) A polynucleotide including a variant of the nucleotide sequence according to one of SEQ ID NO.1 to SEQ ID NO:9, wherein the polynucleotide forms a nucleic acid ligand that identifies at least one difference at the molecular level between two complex biological mixtures. In this regard, the polynucleotide forms a nucleic acid ligand that identifies at least one difference at the molecular level between a malignant mesothelioma cell and a non-malignant mesothelial cell;
(iii) A polynucleotide that hybridises with the complement of the nucleotide sequence according to one of SEQ ID NO.1 to SEQ ID NO:9 under stringent hybridisation conditions, wherein the polynucleotide forms a nucleic acid ligand that identifies at least one difference at the molecular level between two complex biological mixtures. In this regard, stringent hybridisation conditions include hybridisation in 4×SSC at 65° C. and washing in 0.1×SSC at 65° C. or hybridisation in 50% formamide, 5×SSC and 1% SDS at 65° C. and washing in 0.2×SSC and 0.1% SDS at 65° C.;
(iv) A nucleic acid ligand including a nucleotide sequence according to one of SEQ ID NO:1 to SEQ ID NO:9, wherein the ligand distinguishes a malignant mesothelioma cell and the non-malignant cell from a non-malignant mesothelial cell;
(v) A method of identifying at least one difference at the molecular level between a first complex biological mixture and a second complex biological mixture, the method including the steps of:
(a) binding to a first complex biological mixture a nucleic acid ligand including the nucleotide sequence of one of SEQ ID NO:1 to SEQ ID NO:9, or a variant thereof;
(b) binding to a second complex biological mixture a nucleic acid ligand including the nucleotide sequence of one of SEQ ID NO:1 to SEQ ID NO:9, or a variant thereof; and
(c) identifying at least one difference at the molecular level between the first complex biological mixture and the second complex biological mixture by the differential binding of the nucleic acid ligand to the first complex biological mixture and the second biological mixture; and
(vi) A method of identifying a malignant mesothelioma cell, the method including the steps of:
(a) binding to a test cell or cellular extract a nucleic acid ligand including the nucleotide sequence of one of SEQ ID NO:1 to SEQ ID NO:9, or a variant thereof;
(b) binding to a non-malignant mesothelial cell or cellular extract a nucleic acid ligand including the nucleotide sequence of one of SEQ ID NO:1 to SEQ ID NO: 9, or a variant thereof; and
(c) identifying the test cell as a malignant mesothelioma cell by differential binding of the nucleic acid ligand to the test cell or cellular extract and the non-malignant cell or cellular extract.

Nucleic acid ligands with the nucleotide sequence according to SEQ ID NO: 10 through SEQ ID NO:27 are useful for distinguishing malignant prostate cells from non-malignant prostate cells.

As such the present invention contemplates the following further forms of the present invention:
(i) A polynucleotide including the nucleotide sequences according to one of SEQ ID NO:10 to SEQ ID NO:27;
(ii) A polynucleotide including a variant of the nucleotide sequence according to one of SEQ ID NO.1o to SEQ ID NO:27, wherein the polynucleotide forms a nucleic acid ligand that identifies at least one difference at the molecular level between two complex biological mixtures. In this regard, the polynucleotide forms a nucleic acid ligand that identifies at least one difference at the molecular level between a malignant prostate cell and a non-malignant prostate cell;
(iii) A polynucleotide that hybridises with the complement of the nucleotide sequence according to one of SEQ ID NO.10 to SEQ ID NO:27 under stringent hybridisation conditions, wherein the polynucleotide forms a nucleic acid ligand that identifies at least one difference at the molecular level between two complex biological mixtures. In this regard, stringent hybridisation conditions include hybridisation in 4×SSC at 65° C. and washing in 0.1×SSC at 65° C. or hybridisation in 50% formamide, 5×SSC and 1% SDS at 65° C. and washing in 0.2×SSC and 0.1% SDS at 65° C.;
(iv) A nucleic acid ligand including the sequence of one of SEQ ID NO:10 to SEQ ID NO:27 wherein the ligand distinguishes a malignant prostate cell from a non-malignant prostate cell;
(v) A method of identifying at least one difference at the molecular level between a first complex biological mixture and a second complex biological mixture, the method including the steps of:
(a) binding to a first complex biological mixture a nucleic acid ligand including the nucleotide sequence of one of SEQ ID NO:10 to SEQ ID NO:27, or a variant thereof;
(b) binding to a second complex biological mixture a nucleic acid ligand including the nucleotide sequence of one of SEQ ID NO:10 to SEQ ID NO:27, or a variant thereof; and
(c) identifying at least one difference at the molecular level between the first complex biological mixture and the second complex biological mixture by the differential binding of the nucleic acid ligand to the first complex biological mixture and the second biological mixture.
(vi) A method of identifying a malignant prostate cell, the method including the steps of:
(a) binding to a test cell or cellular extract a nucleic acid ligand including the nucleotide sequence of one of SEQ ID NO:10 to SEQ ID NO:27, or a variant thereof;
(b) binding to a non-malignant prostate cell or cellular extract a nucleic acid ligand including the nucleotide sequence of one of SEQ ID NO:10 to SEQ ID NO: 27, or a variant thereof; and
(c) identifying the test cell as a malignant prostate cell by differential binding of the nucleic acid ligand to the test cell or cellular extract and the non-malignant cell or cellular extract.

Nucleic acid ligands with the nucleotide sequence according to SEQ ID NO: 28 through SEQ ID NO:32 are useful for distinguishing malignant adenoma cells from non-malignant bowel cells.

As such the present invention contemplates the following further forms of the present invention:
(i) A polynucleotide including the nucleotide sequences according to one of SEQ ID NO:28 to SEQ ID NO:32;
(ii) A polynucleotide including a variant of the nucleotide sequence according to one of SEQ ID NO.28 to SEQ ID NO:32, wherein the polynucleotide forms a nucleic acid ligand that identifies at least one difference at the molecular level between two complex biological mixtures. In this regard, the polynucleotide forms a nucleic acid ligand that identifies at least one difference at the molecular level between a malignant adenoma cell and a non-malignant bowel cell;
(iii) A polynucleotide that hybridises with the complement of the nucleotide sequence according to one of SEQ ID NO.28 to SEQ ID NO:32 under stringent hybridisation conditions, wherein the polynucleotide forms a nucleic acid ligand that identifies at least one difference at the molecular level between two complex biological mixtures. In this regard, stringent hybridisation conditions include hybridisation in 4×SSC at 65° C. and washing in 0.1×SSC at 65° C. or hybridisation in 50% formamide, 5×SSC and 1% SDS at 65° C. and washing in 0.2×SSC and 0.1% SDS at 65° C.;
(iv) A nucleic acid ligand including the sequence of one of SEQ ID NO:28 to SEQ ID NO:32 wherein the ligand distinguishes a malignant adenoma cell from a non-malignant bowel cell;
(v) A method of identifying at least one difference at the molecular level between a first complex biological mixture and a second complex biological mixture, the method including the steps of:
(a) binding to a first complex biological mixture a nucleic acid ligand including the nucleotide sequence of one of SEQ ID NO:28 to SEQ ID NO:32, or a variant thereof;
(b) binding to a second complex biological mixture a nucleic acid ligand including the nucleotide sequence of one of SEQ ID NO:28 to SEQ ID NO:32, or a variant thereof; and
(c) identifying at least one difference at the molecular level between the first complex biological mixture and the second complex biological mixture by the differential binding of the nucleic acid ligand to the first complex biological mixture and the second biological mixture.
(vi) A method of identifying a malignant adenoma cell, the method including the steps of:
(a) binding to a test cell or cellular extract a nucleic acid ligand including the nucleotide sequence of one of SEQ ID NO:28 to SEQ ID NO:32, or a variant thereof;
(b) binding to a non-malignant bowel cell or cellular extract a nucleic acid ligand including the nucleotide sequence of one of SEQ ID NO:28 to SEQ ID NO: 32, or a variant thereof; and
(c) identifying the test cell as a malignant adenoma cell by differential binding of the nucleic acid ligand to the test cell or cellular extract and the non-malignant cell or cellular extract.

With regard to the nucleic acid ligand including the nucleotide sequence of SEQ ID NO:1, examples of malignant cells that may be distinguished by this nucleic acid ligand from non-malignant cells include (i) malignant mesothelioma cells (including epithelioid mesothelioma cells, biphasic mesothelioma cells, desmoplastic mesothelioma cells and sarcomatoid mesothelioma cells) and normal lung cells or benign or reactive mesothelial cells; (ii) malignant lung cells (including lung adenocarcinoma cells, lung small cell carcinoma cells, lung large carcinoma cells and lung squamous cell carcinoma cells) and non-malignant lung cells; (iii) malignant bowel cells (bowel adenoma cells and bowel carcinoma cells) and non-malignant bowel cells; and (iv) malignant prostate cells and non-malignant prostate cells.

The ability of the nucleic acid ligand to distinguish between a malignant cell and a non-malignant cell may be confirmed by exposing the nucleic acid ligand under the appropriate conditions to one or more malignant and non-malignant cells and detecting the extent of differential binding of the nucleic acid ligand to the malignant and non-malignant cells.

For example, for distinguishing between malignant mesothelioma cells and non-malignant mesothelial cells, formalin fixed tissue sections may be used. In this case, the sections may be de-paraffinised and washed through a series of graded alcohol before undergoing antigen retrieval (121° C. in sodium citrate buffer pH 6.5 for 12 min, then left to cool for 2 hrs). The antigen retrieved tissue sections may then be equilibrated in binding buffer (1×PBS, 5 mM MgCl₂) and incubated overnight in a humidified chamber with thermally equilibrated nucleic acid ligand (1 nM). The sections may then be thoroughly washed in binding buffer to remove unbound ligand and the ligand detected. An Enzyme Labelled Fluorescence (ELF) kit (Molecular Probes, USA) is suitable for this purpose. In this instance, the biotinylated ligand is bound to streptavidin which is bound to alkaline phosphatase that reacts with the ELF substrate. This reaction produces an intensely fluorescent yellow green precipitate at the site of enzymatic activity.

Accordingly, in a preferred form the present invention provides a nucleic acid ligand that distinguishes a malignant mesothelioma cell from a non-malignant mesothelial cell.

A similar procedure is also suitable for distinguishing malignant lung cells (including lung adenocarcinoma cells, lung small cell carcinoma cells, lung large carcinoma cells and lung squamous cell carcinoma cells) from non-malignant lung cells, malignant bowel cells (bowel adenoma cells and bowel carcinoma cells) from non-malignant bowel cells, and malignant prostate cells from non-malignant prostate cells.

Accordingly, in another preferred form the present invention provides a nucleic acid ligand that distinguishes a malignant lung cell from a non-malignant lung cell.

In a further preferred form, the present invention provides a nucleic acid ligand that distinguishes a malignant bowel cell from a non-malignant bowel cell.

In another preferred form, the present invention provides a nucleic acid ligand that distinguishes a malignant prostate cell from a non-malignant prostate cell.

As described previously, the nucleic acid ligand may be synthesized, purified and isolated by a method known in the art. For example, phosphorothioate polynucleotides may be synthesized by the method as described in Stein et al. (1988) Nucl. Acids Res. 16: 3209.

The present invention also provides a method of identifying at least one difference at the molecular level between a first complex biological mixture and a second complex biological mixture, the method including the steps of:
(a) binding to a first complex biological mixture a nucleic acid ligand including the nucleotide sequence of SEQ ID NO:1 or a variant thereof;
(b) binding to a second complex biological mixture a nucleic acid ligand including the nucleotide sequence of SEQ ID NO:1 or a variant thereof; and
(c) identifying at least one difference at the molecular level between the first complex biological mixture and the second complex biological mixture by the differential binding of the nucleic acid ligand to the first complex biological mixture and the second biological mixture.

Preferably, the first complex biological mixture is a first cell or an extract thereof, and the second biological system is a second cell or an extract thereof.

The first and second cells may be present in a tissue sample such as a formalin fixed tissue sample, a biopsy or a blood sample. Alternatively, the first and second cells may be present as cells maintained or propagated in culture, or may be cells present in an entire animal or human.

More preferably, the first complex biological mixture is a cell in a formalin fixed tissue sample and the second complex biological mixture is a cell in a formalin fixed tissue sample.

Preferably, the first complex biological mixture is a malignant cell or an extract thereof, and the second cell is a non-malignant cell or an extract thereof. For example, the malignant cell may be a malignant mesothelioma cell (including an epithelioid mesothelioma cell, a biphasic mesothelioma cell, a desmoplastic mesothelioma cell or a sarcomatoid mesothelioma cell) and the non-malignant cell be a normal, benign or reactive mesothelial cell. Alternatively, the malignant cells may be a lung cell (including a lung adenocarcinoma cell, a lung small cell carcinoma cell, a lung large carcinoma cell or a lung squamous cell carcinoma cell) and the non-malignant cell a non-malignant lung cell, or the malignant cell may be a malignant bowel cell (including a bowel adenoma cell or bowel carcinoma cell) and the non-malignant cell a non-malignant bowel cell; or the malignant cell may be a malignant prostate cell and the non-malignant cell a non-malignant prostate cell.

Accordingly, in a preferred form the present invention provides a method of identifying a malignant cell, the method including the steps of:
(a) binding to a test cell or cellular extract a nucleic acid ligand including the nucleotide sequence of SEQ ID NO:1 or a variant thereof;
(b) binding to a non-malignant cell or cellular extract a nucleic acid ligand including the nucleotide sequence of SEQ ID NO:1 or a variant thereof; and
(c) identifying the test cell as a malignant cell by differential binding of the nucleic acid ligand to the test cell or cellular extract and the non-malignant cell or cellular extract.

The binding of the nucleic acid ligand to the first and second cells or cellular extracts may be performed under conditions suitable known in the art to allow the nucleic acid ligand to detect at least one difference between the cells.

For example, for distinguishing between malignant mesothelioma cells and non-malignant mesothelial cells, formalin fixed tissue sections may be used. In this case, the sections may be de-paraffinised and washed through a series of graded alcohol before undergoing antigen retrieval (121° C. in sodium citrate buffer pH 6.5 for 12 min, then left to cool for 2 hrs). The antigen retrieved tissue sections may then be equilibrated in binding buffer (1×PBS, 5 mM MgCl₂) and incubated overnight in a humidified chamber with thermally equilibrated nucleic acid ligand (1 nM). The sections may then be thoroughly washed in binding buffer to remove unbound ligand and the ligand detected. An Enzyme Labelled Fluorescence (ELF) kit (Molecular Probes, USA) is suitable for this purpose. In this instance, the biotinylated ligand is bound to streptavidin which is bound to alkaline phosphatase that reacts with the ELF substrate. This reaction produces an intensely fluorescent yellow green precipitate at the site of enzymatic activity.

A similar procedure is also suitable for distinguishing malignant lung cells (including lung adenocarcinoma cells, lung small cell carcinoma cells, lung large carcinoma cells and lung squamous cell carcinoma cells) from non-malignant lung cells, malignant bowel cells (bowel adenoma cells and bowel carcinoma cells) from non-malignant bowel cells, and malignant prostate cells from non-malignant prostate cells.

As discussed previously, the nucleic acid ligand may be detectably labelled by a method known in the art. For example, the nucleic acid ligand may be labelled with biotin and the ligand detected by way of a biotin:streptavidin complex.

The present invention further contemplates the use of the various nucleic acid ligands as diagnostic agents, as therapeutic agents, or as carriers for therapeutic agents, for the treatment of various diseases, conditions and states. For example, the nucleic acid ligands of the present invention may be useful for the diagnosis and/or treatment of various diseases conditions, and states of the mesothelium, lungs, pleura, bowel, prostate and blood, various degenerative diseases, including degenerative diseases of the eye, various cancers including cancers of mesothelium, lungs, pleura, bowel, prostate and blood (eg leukaemia), and for the diagnosis and/or treatment of various bacterial or viral infections, or diseases or conditions associated with such bacterial or viral infections. The present invention also contemplates the use of the various nucleic acid ligands as reagents for imaging for diagnostic purposes.

The present invention also contemplates the use of the nucleic acid ligands as tools for identification of their target molecules in complex mixtures. For example, by the use of affinity chromatography it may be possible to identify the various protein and non-protein targets of the ligands in cells.

The present invention also contemplates the use of the nucleic acid ligands as tools for the identification and/or isolation of various cell types, such as stem cells, fetal erythrocytes, trophoblasts and other rare or difficult to identify/isolate cell types. For example, the ligands may be labelled so as to allow FACS analysis of various cell types.

### DESCRIPTION OF PREFERRED EMBODIMENTS

The present invention will now be described in relation to various examples of preferred embodiments. However, it must be appreciated that the following description is not to limit the generality of the above description.

### EXAMPLE 1

The following example relates to the isolation of a pool of nucleic acid ligands capable of differentiating between normal liver tissue and cancerous tissue.

Formalin fixed human tissue sections of colon tumour metastases in liver were prepared. Colon tumour metastases were identified in the liver tissue by standard histopathological procedures. A tissue section in which the tumourigenic tissue represented less than 10% of the total cell population in each section was selected.

A 10 micrometer thick tissue section was deposited on a glass slide and antigen retrieval performed by microwave irradiation of the tissue sample followed by ribonuclease A treatment.

One to fifty micrograms of a chemically random synthesised aptamer library of average size of 85 nucleotides containing a randomised section of 45 bases (2×10¹³ molecules per microgram) in 0.2 ml binding buffer (0.15 M NaCl, 10 mM phosphate pH 7.4, 5 mM MgCl₂) was used. One million counts per minute of radioactively labeled library were also included in the sample for the purpose of monitoring the final binding of the aptamer library to the target tumourigenic tissue.

The aptamer library was heat denatured and allowed to slowly cool to room temperature over a period of thirty minutes. The library solution was then placed on the surface of the tissue section and allowed to incubate at room temperature for 4 hours in a humidified container.

The tissue section was washed six times with five ml of binding buffer to remove unbound aptamers and the tissue section placed under a microsocpe and the tumourigenic target cell population recovered by scraping with a scalpel or a fine needle. Total nucleic acids were extracted and nucleic acids purified from the recovered tissue by using a standard guanidine thiocyanate, acid phenol and alcohol precipitation isolation procedure.

To determine the proportion of aptamer bound to the tumourigenic tissue, 1% of the recovered nucleic acid was taken and the amount of radioactive material determined by scintillation counting.

Single stranded DNA was amplified by PCR using standard procedures. Complementary DNA strands were separated by non-denaturing polyacrylamide gel electrophoresis and the DNA strands recovered from the gel by electroelution.

If the aptamer library used was a RNA-based aptamer library, the RNA aptamers were first converted to cDNA with reverse transcriptase using standard protocols before amplification. To regenerate RNA ligands for re-binding to the target, in vitro transcription was utilised from the amplified pool. Alternatively, the amplified products was cloned into a vector and the library of inserts then transcribed in vitro to regenerate the RNA ligands.

At this point the aptamer library was rebound to similar tissue sections and the process repeated. Cycles of the process were repeated until the amount of radioactively labeled nucleic acids binding to the target cell population reached a plateau.

The double stranded DNA resulting from the final round of selection was cloned into a plasmid vector (for example pGEM T Easy from Promega) using E. coli DH5α as a hosts. The total plasmid DNA was isolated and the library of inserts amplified by PCR using one biotinylated primer and a normal primer. The resulting biotinylated strands were used to veryify by staining of tissue sections that the pool of aptamers so isolated showed an increased signal to the tumourigenic tissue over the normal tissue in the tissue sample.

This was done by taking 1 to 10 micrograms of the biotinylated aptamer and incubating with a new tissue section under exactly the same conditions that were used in its isolation. Unbound aptamer was washed from the section and the sites of aptamer binding visualized using a streptavidin-horseradish peroxidase complex and a standard enzyme substrate.

In addition, individual clones were randomly picked and the inserts amplified by PCR using one biotinylated oligonucleotide and one normal oligonucleotide. The resulting biotinylated strands were then purified by denaturing polyacrylamide gel electrophoresis and the specificity of aptamer binding was determined by taking 1 to 10 micrograms of the biotinylated aptamer and incubating with a new tissue section under exactly the same conditions that were used in its isolation. Unbound aptamer was washed from the section and the sites of aptamer binding visualized using a streptavidin-horseradish peroxidase complex and a standard enzyme substrate. The apatmers so recovered showed specific binding to the target cell population and only background binding to other regions.

Additional rounds of apatmer selection to remove background can be undertaken using sections from other non-target tissues.

Affinity of the aptamer population and or individual aptamers can be further enhanced by performing mutagenesis on the selected aptamer pool followed by selection on target tissue sections as described.

### EXAMPLE 2

The following example relates to the isolation of a pool of individual aptamers that bind to specific molecules present in serum.

Serum proteins were concentrated and partially enriched by ammonium sulfate precipitation. The protein mixture was desalted by dialysis. Proteins were then immobilized on activated CH-Sepharose (Pharmacia) using conditions recommended by the supplier. Populations of beads were created with protein content varying between 1 and 25 microgram of protein per milligram of beads.

Alternatively the protein mixture was biotinylated with EZ-Link-sulfo-NH S-LC-Biotin (Pierce) which primarily reacts with free amino groups of lysine residues.

10-50 micrograms of single stranded DNA aptamer library (>1×10¹⁴ molecules) was spiked with ³²P end labelled library (1×10⁵ CPM) was thermally equilibrated in binding buffer then added to underivatized CH— Sepharose to remove Sepharose binding species. The mixture was incubated at room temperature for 1.5 hours with constant agitation.

Unbound aptamers were recovered by centrifugation and then added to protein coupled CH-Sepharose. The mixture was incubated at room temperature for 1.5 hours with constant agitation.

Uncoupled and protein coupled beads were washed 4 times in binding buffer. The amount of radioactivty associated with the washes was determined, and the counts associated with a portion of the protein coupled CH-Sepharose were determined by scintillation counting.

Aptamers bound to protein were eluted in 7M urea with heating and recovered by ethanol precipitation. Recovered aptamers were then subject to PCR amplification using oligonucleotides to the common flanking regions. One oligonucleotide was biotinylated to facilitate strand separation.

Amplified products were pooled, ethanol precipitated and then incubated with streptavidin to bind to the biotin The streptavidin:biotin:DNA complex was then subject to denaturing polyacrylamide gel electrophoresis. Under these conditions, the non-biotinylated strand migrates ahead of the streptavidin:biotin:DNA complex.

Gels were stained with SYBr Gold (Molecular Probes) and the single stranded DNA visualised using a Fluorimager (Molecular Dynamics). Single stranded DNA was recovered from the gel by electroelution. Eluted species were purified and concentrated by phenol/chloroform extraction and ethanol precipitation and quantitated using SYBr Green II stain on a Fluorimager.

The aptamer population resulting from the first round of selection was cloned into a vector pGEM-T Easy (Promega) and 100 individual clones isolated and sequenced. The inserts from each of these clones was amplified by PCR using one oligonucleotide phosphorylated at the 5′ end and one oligonucleotide with a primary amine at the 5′ end. The DNA strand containing the phosphorylated 5′ end was degraded by incubating the PCR product with lambda exonuclease under standard conditions. The remaining single DNA strand, corresponding to the original aptamer sequence, was purified by standard phenol/chloroform extraction and ethanol precipitation.

The single stranded DNA was then coupled to a solid support of microspheres using established methods. Each aptamer was coupled to microspheres containing a unique addressable optical code based on Qdot nanocrystals (Quantum Dot Corporation).

An aliquot of the protein target mixture was then incubated with each immobilized aptamer and unbound proteins removed by washing in binding buffer.

Specifically bound proteins were eluted from the immobilized aptamer using binding buffer containing 6M urea or 0.5% sodium dodecylsulfate. An aliquot of this eluate was then analyzed by MALDI-TOF mass spectrometry using a Bruker Autoflex instrument.

The identity of each protein eluate was then assigned by mass values obtained from the mass spectral trace. Each aptamer was then classified according to its binding specificity.

Aptamers shown to bind a single protein were then produced in large quantity either by solid phase synthesis or as described above and immobilized on a solid support as described.

The original target protein mixture was then passed over this population of aptamers to remove proteins identified in the first round of selection.

Proteins which did not bind to these aptamers were then used for the second round of aptamer selection and protein identification. Repeated rounds of aptamer selection and protein identification will eventually allow isolation of an aptamer to and identification of every protein in the mixture.

Aptamers produced in this manner may then be incorporated into a diagnostic format that will allow the concentration of every protein in the target mixture to be determined. In addition the aptamers could be used to tag individual proteins for therapeutic or diagnostic purposes.

### EXAMPLE 3

Preparation of Aptamer Library

An 85 mer with a 45 base section of random nucleotide sequence was synthesized. The nucleotide sequence of the 85 mer is as follows:

5′-AGCTCAGAATAAACGCTCAANNNNNNNNNNNNNNNNNNNNNNNNNN
NNNNNNNNNNNNNNNNNNNTTCGACATGAGGCCCGGATC-3′

The 85 mer was dissolved in water to a concentration of approximately 100 μM.

To generate a biotin labelled aptamer for use in screening, aN oligonucleotide with the following sequence was synthesized:

5′-GATCCGGGCCTCATGTCGAA-3′

This oligonucleotide was dissolved in water to a concentration of 100 μM. To anneal the oligonucleotide to the 85 mer, 25 μl of 100 μM 85-mer was mixed with 10 μl of 100 uM oligonucleotide, 30 μl Sequenase buffer (USB; 5×) and 94 μL water. The reaction was mixed and incubated at 68° C. for 5 minutes, the mix cooled to room temperature for 5 minutes and then chilled on ice for 2 minutes.

To the above mix was added 16.5 μl 0.1 M DTT, 12.5 μl 10 mM dNTPs, 1 μl Sequenase (USB, 13 μl/μl), 20 μl 5× Sequenase buffer and 41 μl H₂O. The reaction was incubated at 42° C. for 30 minutes. A 8% PAGE was run to assess end-filling.

The end-filled reaction mix was then heat inactivated at 65° C. for 15 minutes, cooled to room temperature and 1.5 μl Exonuclease l (20 μl/μl) added. The reaction was incubated at 37° C. for 30 minutes and then heat inactivated at 80° C. for 15 minutes.

The mix was phenol:CHCl₃ extracted and the DNA ethanol precipitated. The amount of DNA was quantitated.

To generate biotin labelled aptamer for screening, 25 ng of dsDNA was combined in a 100 μl reaction with 1-2 units Taq polymerase, 10 μl 1×Taq buffer, 2 μl 100 mM MgSO₄, 2 μl 10 mM dNTPs, 30 pmol of the oligonucleotide as above and 30 pmol of a biotin-labelled primer with the sequence as follows:

5′-GGGAGCTCAGAATAAACGCTCAA-3′,

5 to 8 PCR cycles are sufficient to amplify sufficient product for screening. The biotin labelled aptamer is recovered by running on a 6% denaturing PAGE gel and excising the aptamer.

### EXAMPLE 4

Generation of Individual Aptamers

A single colony was picked into 25 μl of lysis buffer (20 mM EDTA, 2 mM Tris pH 8.5, 1% Triton x-100). The colony was lysed by heating at 99° C. for 10 minutes, and then stored until ready at 4° C.

To 1 μl of the cracked colony was mixed with 19 μl of M13 buffer (a master mix prepared by mixing 50 μl 10×Taq buffer, 10 μl 100 mM MgSO₄, 10 μl 10 mM dNTPs, 10 μl 10 μM M13 forward primer, 10 μl 10 μM M13 reverse primer, 2.5 μl Taq polymerase (2μ/μl) and 382.5 μl H₂O).

To 2.5 μl of the above mix was added 7.5 μl of Exol mix (a master mix was prepared by mixing 3 μl Exol (20 μ/μl), 22.5 μl 10×Exol buffer (New England Biolabs) and 199.5 μl H₂O). The mixture was incubated at 37° C. for 15 minutes and then heated at 80° C. for 15 minutes.

To prepare biotinylated aptamer, 1 μl of the Exol treated PCR was added to 100 μl of primer cocktail (prepared by mixing 500 μl 10×NEB buffer, 100 μl 10 mM dNTPs, 10 μl 100 uM biotinylated primer, 10 μl 100 uM phosphorylated primer, 5 μl Taq polymerase (NEB 5 μ/μl and 4375 μl H₂O). The reaction mix was split into two 100 μl aliquots and 25 cycles of PCR performed and the samples pooled.

2 μl of Exol mix (6 μl Exol (20 μ/μl NEB) 6 μl 10×Exol buffer and 48 μl H₂O) was added to the 200 μl PCR reaction. The mix was incubated for 20 minutes at 37° C., extracted with 50 μl CHCl₃, recovery of the aqueous phase (approx 180 μl), 18 μl 3M Na acetate pH 5.2 added, followed by 450 μl ethanol. The DNA was precipitated for 60 minutes at −20° C., spun in an eppendorf centrifuge for 15 minutes at room temperature, air-dried and resuspended in 50 μl H₂O. The amount of DNA was quantitated with a picogreen assay.

Aptamers were then purified on a 8% PAGE gel.

### EXAMPLE 5

Production of an Aptamer (MTA R72) that Detects Malignant Mesothelioma

Malignant mesothelioma of the pleura was used as a model system for the ability to isolate aptamers that detect malignant versus benign reactive mesotheliosis and/or fibrous pleuritis.

The differential diagnosis of malignant mesothelioma versus benign reactive mesotheliosis and/or fibrous pleuritis is a difficult diagnosis to make, both clinically and histologically. Whilst antibodies help to distinguish mesothelioma from adenocarcinoma, the diagnosis of benign mesotheliosis and malignant mesothelioma typically requires considerable expertise on the part of the pathologist who is reliant on a panel of antibodies and accurate clinical and radiological information. However, in some cases a definite conclusion still cannot be made and only clinical follow up will render the final diagnosis.

(i) Mesothelioma Tissues

Cases of malignant mesothelioma were retrieved from the files of the Department of Anatomical Pathology, Flinders Medical Centre. All cases had been diagnosed by an expert in pleural pathology (Douglas W. Henderson, Flinders Medical Centre, Adelaide) by employing light microscopy, a panel of monoclonal antibodies routinely used in the laboratory for the differentiation between mesothelioma and adenocarcinoma (essentially as described in Moran, C. A., M. R. Wick, and S. Suster (2000) “The role of immunohistochemistry in the diagnosis of malignant mesothelioma” Semin Diagn Pathol. 17(3): p. 178-83, and in Ordonez, N. G. (2002) “Immunohistochemical diagnosis of epithelioid mesotheliomas: a critical review of old markers, new markers” Hum Pathol. 33(10): p. 953-67), and electron microscopy in selected cases. All cases were reviewed for adequacy of the tissue in the block. There were 18 cases of malignant mesothelioma, consisting of 11 epithelioid mesotheliomas, 4 sarcomatoid/desmoplastic mesotheliomas and 3 biphasic mesotheliomas. Also, 5 cases of benign mesotheliosis/fibrous pleuritis were included as negative controls.

(ii) Generating Aptamers as Histological Markers

An oligonucleotide library was synthesised commercially containing 45 random nucleotides. A starting pool of 10¹⁴ oligonucleotides was screened in the first round of selection.

One to fifty micrograms of a chemically random synthesised aptamer library of average size of 85 nucleotides containing a randomised section of 45 bases (2×10¹³ molecules per microgram) in 0.2 ml binding buffer (0.15 M NaCl, 10 mM phosphate pH 7.4, 5 mM MgCl₂) was used. One million counts per minute of radioactively labeled library were also included in the sample for the purpose of monitoring the final binding of the aptamer library to the target tumourigenic tissue.

The aptamer library was heat denatured and allowed to slowly cool to room temperature over a period of thirty minutes. The library solution was then placed on the surface of the tissue section and allowed to incubate at room temperature for 4 hours in a humidified container.

The tissue section was washed six times with five ml of binding buffer to remove unbound aptamers and the tissue section placed under a microsocpe and the tumourigenic target cell population recovered by scraping with a scalpel or a fine needle. Total nucleic acids were extracted and nucleic acids purified from the recovered tissue by using a standard guanidine thiocyanate, acid phenol and alcohol precipitation isolation procedure.

To determine the proportion of aptamer bound to the tumourigenic tissue, 1% of the recovered nucleic acid was taken and the amount of radioactive material determined by scintillation counting.

Single stranded DNA was amplified by PCR using standard procedures. Complementary DNA strands were separated by non-denaturing polyacrylamide gel electrophoresis and the DNA strands recovered from the gel by electroelution.

At this point the aptamer library was rebound to similar tissue sections and the process repeated. Cycles of the process were repeated until the amount of radioactively labeled nucleic acids binding to the target cell population reached a plateau. Typically 5 to 9 rounds were required.

After subsequent rounds of positive selection against the target tissue, individual aptamers were isolated by cloning. Aptamers were screened against their target tissue as described below and selected upon its ability to bind only to the cells of interest

(iii) Detecting Aptamer Bound to its Target

Formalin fixed tissue sections were de-paraffinised in Histo-Clear II (National Diagnostics, USA) and washed through a series of graded alcohol before undergoing antigen retrieval at 121° C. in sodium citrate buffer pH 6.5 for 12 min, then left to cool for 2 hrs. The antigen retrieved tissue sections were equilibrated in Binding Buffer (1×PBS, 5 mM MgCl2) and incubated overnight in a humidified chamber with 1 nM thermally equilibrated aptamer. The sections were thoroughly washed in Binding Buffer to remove unbound aptamer. Aptamer binding was detected using the Enzyme Labelled Fluorescence (ELF) kit (Molecular Probes, USA). Briefly, the biotinylated aptamer is bound to streptavidin which is bound to alkaline phosphatase that reacts with the ELF substrate. This reaction produces an intensely fluorescent yellow green precipitate at the site of enzymatic activity. The sections were counterstained with Harris Haematoxylin for 30 secs before mounting in aqueous medium and coverslipping.

(iv) A Target on All Invasive Malignant Mesothelioma Cells

Using the protocol described above, An aptamer (MTA R72) was isolated that appeared to bind only to malignant mesothelial cells but not to the surrounding stromal tissue.

The nucleotide sequence of MTA R72 was determined from the corresponding clone. The DNA sequence of the aptamer was as follows:

5′-GGGAGCTCAGAATAAACGCTCAAGGAACAGCAAGATACGGTCACCGA
ACATAGCGCACCACAGGCACA-3′.

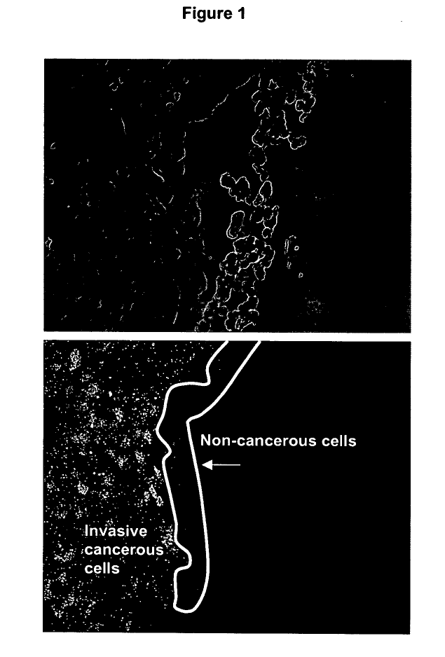

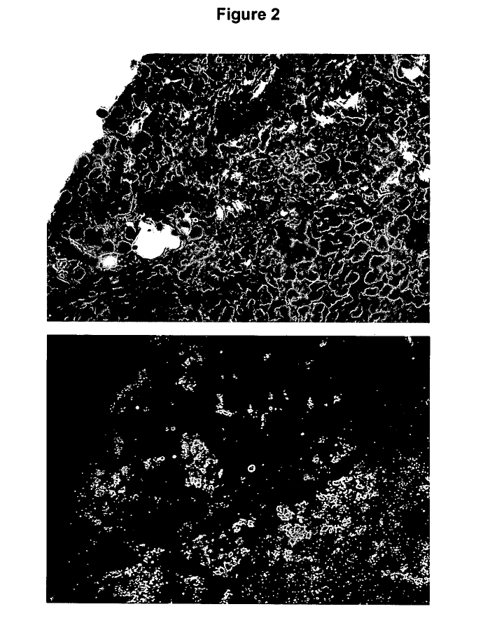

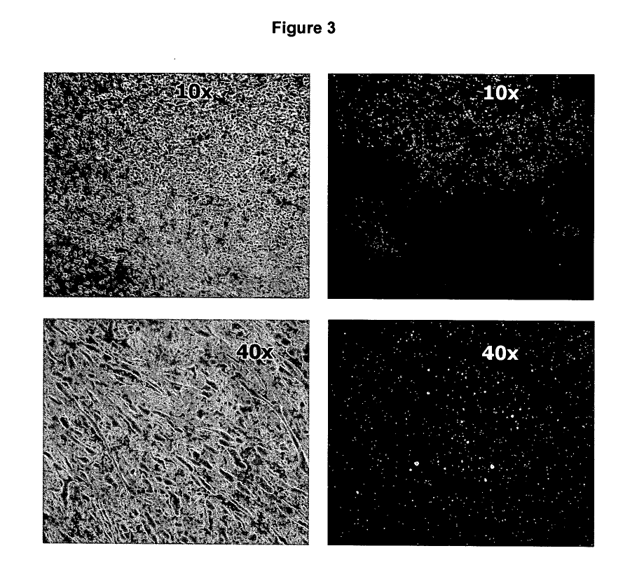

As shown in FIGS. 1 to 3 (both bright and dark fields shown), this aptamer is positive in all cases of malignant mesothelioma and decorates nearly all of the malignant cells, both in the surface and in the invasive component. The staining pattern is predominantly nuclear in epithelioid and biphasic mesothelioma (in both epithelioid and sarcomatoid cells in the latter) as shown in FIGS. 1 and 2. The staining obtained by our fluorescence method is finely granular and clearly apparent at low power (i.e. using a 10×objective) examination. In desmoplastic mesotheliomas, the staining pattern appeared to be cytoplasmic rather than nuclear (FIG. 3), implying that the target is located within the cytoplasm in desmoplastic mesotheliomas, but is nuclear in the other common types of mesothelioma.

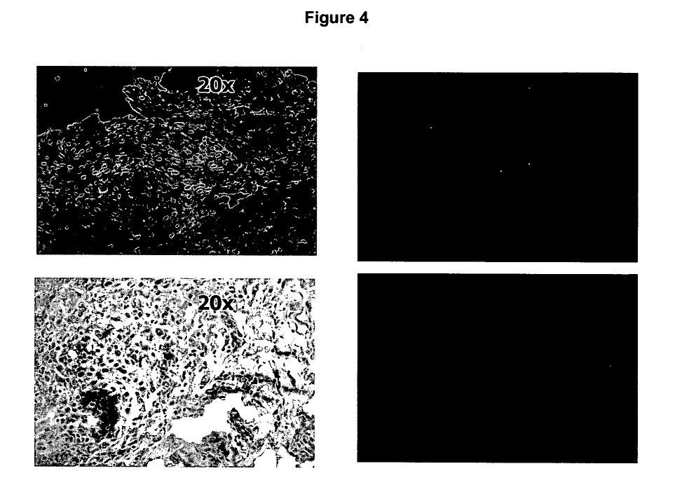

In contrast, no staining was detected in any of the reactive mesotheliosis/fibrous pleuritis cases as shown in FIG. 4.

All of the 18 cases of malignant mesothelioma showed positive labelling with this aptamer whilst none of the 5 reactive/inflammatory mesotheliosis cases exhibited any labelling.

The data presented above show that it is possible to identify aptamers on paraffin-embedded tissue sections that react exclusively with histologically and clinically confirmed malignant mesothelioma tissues, including the epithelioid, desmoplastic/sarcomatoid and biphasic subtypes. One aptamer isolated (MTA R72) detects all cases of mesothelioma tested so far whilst all cases of reactive mesotheliosis/fibrous pleuritis have been negative. The differential diagnosis between mesothelioma and reactive mesothelial proliferations with cytological atypia is often difficult, but this aptamer showed that there are potential targets on malignant cells.

### EXAMPLE 6

Further Studies on the Binding of Aptamer MTA R72 to Mesothelioma Tissue

The binding of aptamer MTA R72 to mesothelioma tissue was also tested for its utility in paraffin based Chromogenic Aptamer HistoChemistry.

In addition, the tissue samples were also tested with IHC using the following antibodies: Calretinin, Cytokeratin 5/6, LCA, and a Negative Control Reagent.

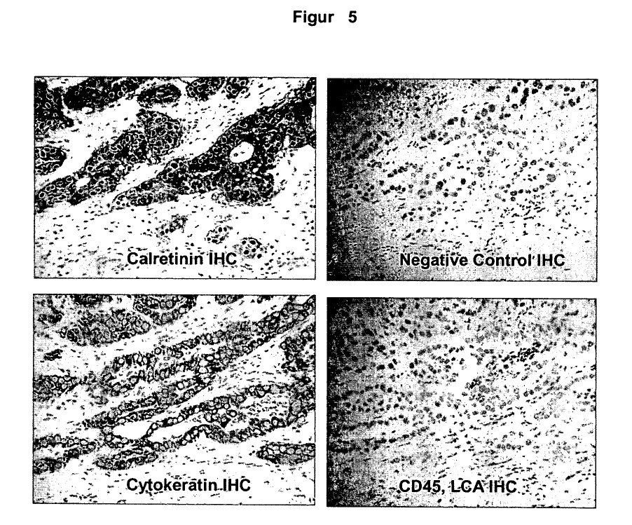

As shown in FIG. 5, Calretinin IHC stained the mesothelioma cells as expected (top left panel). In this case the tissue was pretreated with citrate buffer pH 6.0. IHC-Select Detection with HRP-DAB is shown in brown with Hematoxylin (blue nuclear) counter stain. The Mesothelioma cells stain golden brown, as expected.

The negative control (Diluent for Antibodies) IHC staining of Mesothelioma cells is shown in the top right panel. Tissue was pretreated with Citrate Buffer, pH 6.0. IHC-Select Detection with HRP-DAB is shown in brown with Hematoxylin (blue nuclear) counter stain. No background staining is detected. Cytokeratin 5/6 IHC staining of Mesothelioma is shown in the bottom left panel. Tissue was pretreated with Citrate Buffer, pH 6.0. IHC-Select Detection with HRP-DAB is shown in brown with Hematoxylin (blue nuclear) counter stain. The Mesothelioma cells stain golden brown, as expected.

CD45, LCA IHC staining of Mesothelioma is shown in the bottom right panel. Tissue was pretreated with Citrate Buffer, pH 6.0. IHC-Select Detection with HRP-DAB is shown in brown with Hematoxylin (blue nuclear) counter stain. The Mesothelioma cells do not stain, and only scattered leucocytes stain, as expected.

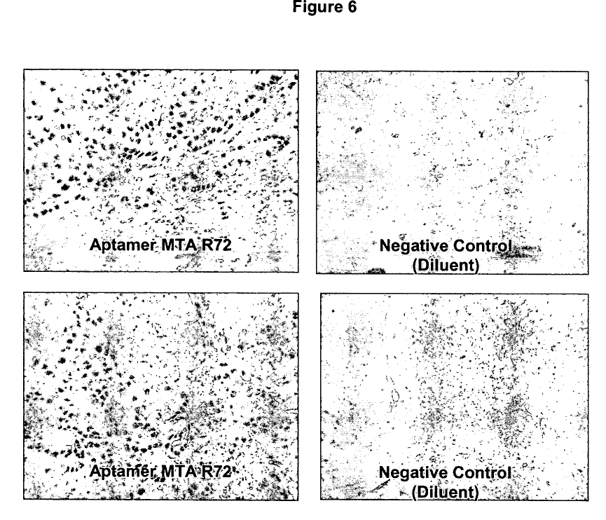

Aptamer MTA-R72 (1 nM) staining of Mesotheliomais shown in FIG. 6, left hand panels. Tissue was pretreated with Citrate Buffer, pH 6.0. Aptamer Detection was with Streptavidin-AP, BCIP/NBT (blue) and Eosin (pink cytoplasmic) counter stain. The Mesothelioma cells stain blue in their nuclei, as expected.

Negative Control (Diluent for Aptamer) staining of Mesothelioma is shown in FIG. 6, right hand panels. Tissue was pretreated with Citrate Buffer, pH 6.0. Aptamer Detection with Streptavidin-AP, BCIP/NBT (blue) and Eosin (pink cytoplasmic) counter stain. No background staining is detected.

These results show that positive staining with MTA-R72 on the Mesothelioma was obtained. Negative controls (omission of the Aptamer from the diluent during the overnight incubation) were clean. In addition, the Calretinin and CK 5/6 antibody stainings were positive, and the LCA and NC were negative (as expected).

These data indicate that light microscopic Chromogenic Aptamer HistoChemistry may also be used for aptamer detection of mesothelioma cells.

### EXAMPLE 7

Aptamer MTA R72 Detects Various Malignant Cell Types

The ability of aptamer MTA R72 to distinguish cells from a variety of different malignant and non-malignant cell types was tested, essentially as described in Example 6. A summary of the results is shown in Table 1.

Summary for APTAMER Mta R7 2

| | | | | StainingPattern | | | NormalCells | | |
|---|---|---|---|---|---|---|---|---|---|
| | Number of | Positive | Negative | | | Nuclear & | | | |
| Tissue/Cancer Description | sections | result | result | Nuclear | Cytoplasm | Cyto. | Epithelium | Stroma | Lymphocytes |
| Mesothelioma, epitheliold | 11 | 11 | 0 | 7 | 1 | 3 | − | − | + |
| Mesothelioma, btphaslc | 3 | 3 | 0 | + | − | − | − | − | + |
| Mesothelioma. desmoplastic | 2 | 2 | 0 | + | − | − | − | − | NA |
| Mesothelioma, sarcomatoid | 2 | 2 | 0 | + | − | − | − | − | NA |
| Adenocarcinoma, lung | 1 | 1 | 0 | + | − | − | − | − | NA |
| Small cell carcinoma, lung | 1 | 1 | 0 | + | − | − | − | − | NA |
| Large cell carcinoma, lung | 1 | 1 | 0 | + | − | − | − | − | NA |
| Squamous cell carcinoma, lung | 1 | 1 | 0 | + | − | − | − | − | NA |
| Adenoma, bowel | 1 | 1 | 0 | + | − | − | − | − | NA |
| Carcinoma, bowel | 1 | 1 | 0 | + | − | − | − | − | NA |
| Prostate cancer | 1 | 1 | 0 | + | − | − | − | − | NA |
| Fibrous pleuritis (negative control) | 2 | 1 | 1 | + | − | − | Query | − | NA |
| Reactive mesotheliosis (neg control) | 3 | 0 | 3 | + | − | − | − | − | + |
| Diffuse atveolar damage (neg control) | 1 | 0 | 1 | + | − | − | − | − | NA |
| Normal lung tissue (neg control) | 1 | 0 | 1 | + | − | − | − | − | NA |

| | | | | | | | | |
|---|---|---|---|---|---|---|---|---|
| Pleural effusion, mesothelioma | 10 | 9 | 1 | + | − | − | Cellsampleonly | NA |
| Pleural effusion, benign conditions | 8 | 1 | 7 | + | − | − | | NA |

NA - information not available due to tissue morphology.

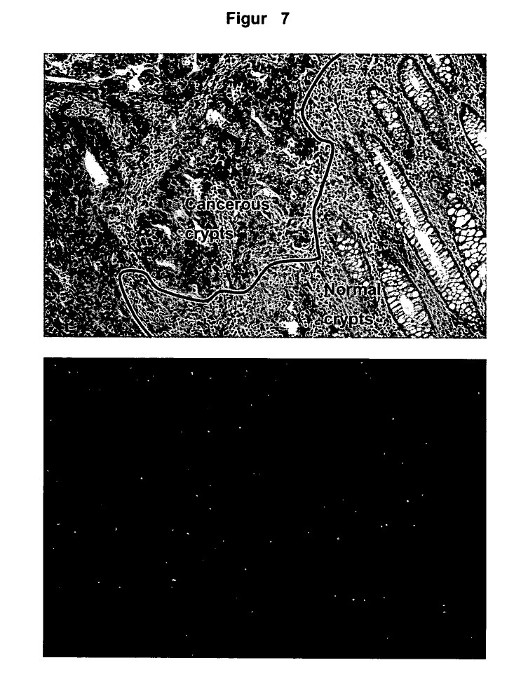

For example, the results of binding of aptamer MTA R72 to bowel carcimona cells is shown in FIG. 7. As can be seen, the colonic adenocarcinoma demonstrates dense punctuate labelling of the invasive glands whilst the benign glands and crypts only show focal “dot-like” staining.

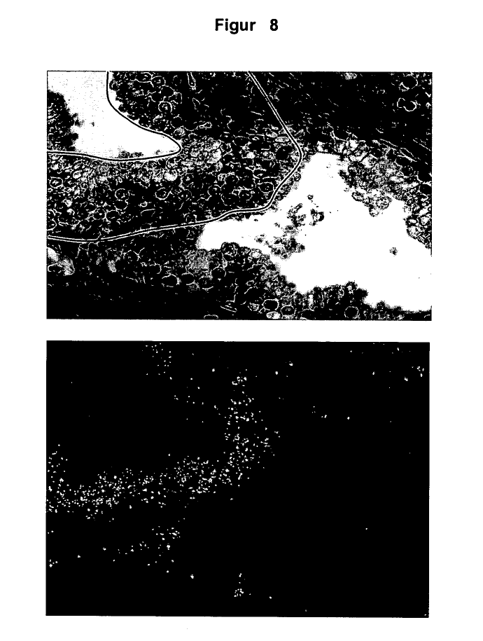

The results of the binding of aptamer MTA R72 to prosate cancer cells is also shown in FIG. 8. Cancerous cells are indicated in the tissue section (left panel) and labelling with the aptamer is shown in the right panel.

### EXAMPLE 7

Aptamers Isolated During Screening of Prostate Cancer Tissue Sections

Screening of prostate cancer tissue sections was performed essentially as described in Example 5.

After 6 rounds of selections, aptamers of the following nucleotide sequence were identified:

| | |
|---|---|
| Prostate R611: | |
| 5′ GGGAGCTCAGAATAAACGCTCAACAATTTTCTTTTTCCCTTCTCTGTCCTTTTCTCC | (SEQ ID NO:10) |
| GTGCTTG3′ | |
| Prostate R612: | |
| 5′ GGGAGCTCAGAATAAACGCTCAACAATTTTCTTTTTCCCTTCTCTGTCCTTTTCTCC | (SEQ ID NO:11) |
| GTGCTTG3′ | |
| Prostate R630 | |
| 5′ GGGAGCTCAGAATAAACGCTCAAGTTTTTCCTCCTGCCTGTTTTCTTCCCCGTGCTC | (SEQ ID NO:12) |
| CTTTTCCCCCC3′ | |
| Prostate R68 | |
| 5′ GGGAGCTCAGAATAAACGCTCAAAAGAATCAGCAGAGACAGGGAGGCGAGAAAGAAG | (SEQ ID NO:13) |
| GGGGGGGGGGAG3′ | |
| Prostate R623 | |
| 5′ GGGAGCTCAGAATAAACGCTCAACAGCCAGGACAGAGAGGTGGGAAACCCAAACAAG | (SEQ ID NO:14) |
| AGCAAATAGCC3′ | |

After 8 rounds of selections, aptamers of the following nucleotide sequence were identified:

| | |
|---|---|
| Prostate R812: | |
| 5′ GGGAGCTCAGAATAAACGCTCAATTGTTTTGGCTTTGTCTCCCGTTTGCCTTCCCCG | (SEQ ID NO:15) |
| GCCTTTGTCTG3′ | |
| Prostate R823: | |
| 5′ GGGAGCTCAGAATAAACGCTCAATCTGGGTCTGTGTGTATCTTTTCCATTGCCTCCT | (SEQ ID NO:16) |
| TCCCTTCGTCT3′ | |
| Prostate R842: | |
| 5′ GGGAGCTCAGAATAAACGCTCAATCTTGCCGGTTCTCTCCTTTTTCCTGTCTGCCTT | (SEQ ID NO:17) |
| CTTTCTCCTTG3′ | |

After 10 rounds of selections, aptamers of the following nucleotide sequence were identified:

| | |
|---|---|
| Prostate R1023 | |
| 5′ GGGAGCTCAGAATAAACGCTCAACCTCCTGTCTGCTCCTATCTCTTGCCTTCCTTGT | (SEQ ID NO:18) |
| TTCCCCTGCC3′ | |
| Prostate R104 | |
| 5′ GGGAGCTCAGAATAAACGCTCAACTCTGTCCTTTCCCTTTCTCCCTTTCTTGCTGCT | (SEQ ID NO:19) |
| CCTTTCGTGTC3′ | |
| Prostate R1046 | |
| 5′ GGGAGCTCAGAATAAACGCTCAACACTTTTCTTGTCCATTTGCTTCTCTACCCTCAT | (SEQ ID NO:20) |
| TCTCCCATCCT3′ | |
| Prostate R1011 | |
| 5′ GGGAGCTCAGAATAAACGCTCAAAGCCTCTCTACCGTGGTGCTGCCCTTCGATTTGT | (SEQ ID NO:21) |
| GTCTGCTGTGT3′ | |
| Prostate R1013 | |
| 5′ GGGAGCTCAGAATAAACGCTCAATGTGGTTTTGCCTTTTCTTTCCGTTTTCTCTTTC | (SEQ ID NO:22) |
| CCTCGCGCCT3′ | |
| Prostate R1010 | |
| 5′ GGGAGCTCAGAATAAACGCTCAACCCCCTGCTTCCCCCTCCTTATGTTGTCCTGCCG | (SEQ ID NO:23) |
| GCGCCTGTACT3′ | |
| Prostate R1020 | |
| 5′ GGGAGCTCAGAATAAACGCTCAATCACTGTCGTCATTTATTTTTTCAGTCCTATTTC | (SEQ ID NO:24) |
| CCTCTCCTGTG3′ | |
| Prostate R1030 | |
| 5′ GGGAGCTCAGAATAAACGCTCAATCCTCTTTTTTGTACGGCCTGCTGTTTGTCTGTG | (SEQ ID NO:25) |
| TGTCTTCCTCA3′ | |
| Prostate R1031 | |
| 5′ GGGAGCTCAGAATAAACGCTCAACCAGTCGGCTCCTTTCCTGCGCGTCTCTTCCCGT | (SEQ ID NO:26) |
| TTTTTCCCCCT3′ | |
| Prostate R1044 | |
| 5′ GGGAGCTCAGAATAAACGCTCAATGTTGCCTAATTCCTGCTATGTTTCTGTCTCCTC | (SEQ ID NO:27) |
| CCCACCCGCGC3′ | |

Of the above, aptamers Prostate R1011, R1013, R1031 and R1045 show various degrees of specific staining of prostate cancer cells as compared to the negative controls.

### EXAMPLE 8

Aptamers Isolated During Screening of Mesothelioma Tissue Sections

Screening of mesothelioma tissue sections was performed essentially as described in Example 5.

After 7 rounds of selections, aptamers of the following nucleotide sequence were identified:

(SEQ ID NO:3)

MTA R78
5′GGGAGCTCAGAATAAACGCTCAAGCCATGGACAAGACTAACGACAGAC
CTAAACCTAAAGGATAAAAA3′

(SEQ ID NO:1)

MTA R72
5′GGGAGCTCAGAATAAACGCTCAAGGAACAGCAAGATACGGTCACCGAA
CATAGCGCACCACAGGCAC3′

(SEQ ID NO:4)

MTA R73
5′GGGAGCTCAGAATAAACGCTCAAACCCGAAAAGCGGGAAAACCCCCAG
CAAATCCCGACCAAAAGCAA3′

(SEQ ID NO:5)

MTA R74
5′GGGAGCTCAGAATAAACGCTCAACCTGTTTTTTTTCCCCCTTATTCTT
CCCCCCCCTGTGTCGC3′

(SEQ ID NO:6)

MTA R75
5′GGGAGCTCAGAATAAACGCTCAATTGTGTGTCTTCTTGCTCTTCTTCC
TTCCCTTGCCTTGCCATTGT3′

(SEQ ID NO:7)

MTA R76
5′GGGAGCTCAGAATAAACGCTCAATGTGTGCTGTCAGGCGTGCTGTGTG
TGTAATCTTGGTGCGGCCTC3′

(SEQ ID NO:8)

MTA R77
5′GGGAGCTCAGAATAAACGCTCAAGCAGGACCAAGAAGACACCCAAAAA
GAAGCAATGAAAGAGGCAG3′

(SEQ ID NO:9)

MTA R71
5′GGGAGCTCAGAATAAACGCTCAAGTTGGGGTGTTGCCTGGTCTGTGTA
CGCTGGGGGCGGTTGCGTGG3′

(SEQ ID NO:2)

MTA R720
5′GGGAGCTCAGAATAAACGCTCAACAAAAGACTATCCAGCGACACGCAA
TCTCAAGCAACAGAGGACAG3′

Of the above, aptamers MTA R72 and MTA R720 showed specific staining of mesothelioma cells as compared to the negative controls. As will be noted, MTA R720 is a variant of MTA R72.

### EXAMPLE 9

Aptamers Isolated During Screening of Bowel Adenoma Tissue Sections

Screening of bowel adenoma tissue sections was performed essentially as described in Example 5.

After 8 rounds of selections, aptamers of the following nucleotide sequence were identified:

(SEQ ID NO:28)

Adenoma R832
5′GGGAGCTCAGAATAAACGCTCAAGCCCCCATAGCAGCAAAGTAAGAAC
AACCAACAGACGCACGACGG3′

(SEQ ID NO:29)

Adenoma R839
5′GGGAGCTCAGAATAAACGCTCAACCAAAAGAACACAACAGAACCAAGC
AGACACCCACACCACCGCAG3′

(SEQ ID NO:30)

Adenoma R846
5′GGGAGCTCAGAATAAACGCTCAAAGTTGGTGTTTTCCTTTCCCTGTCC
CCTTGTTTCATCTTCCCTAC3′

(SEQ ID NO:31)

Adenoma R838
5′GGGAGCTCAGAATAAACGCTCAATCCCTTTTTCCCATCTTTTCGCGGT
TGTTGAGCTTTCTGCGTGTG3′

(SEQ ID NO:32)

Adenoma R834
5′GGGAGCTCAGAATAAACGCTCAAGGACCAGCACACACACCAACAAAGG
CCAGGGACCCGGTACCCACC3′

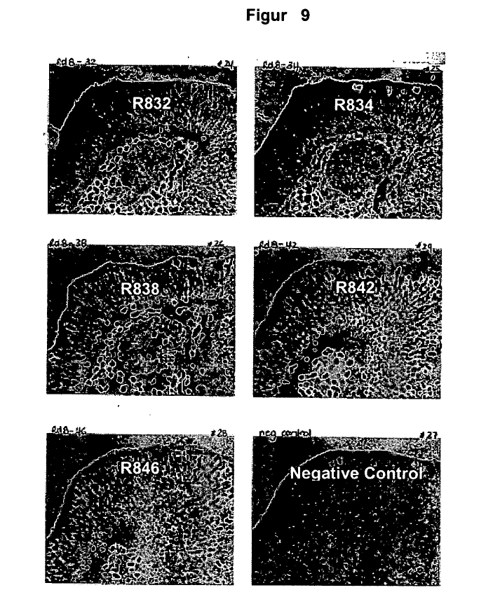

Of the above, aptamers Adenoma R832, R834, R838, R842 (sequence not shown) and R846 and showed some specific staining of adenoma cells as compared to the negative controls, as shown in FIG. 9.

Finally, it will be appreciated that various modifications and variations of the methods, polynucleotides and nucleic acid ligands of the invention described herein will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are apparent to those skilled in the field of molecular biology or related fields are intended to be within the scope of the present invention.

1. A method for isolating a pool of nucleic acid ligands capable of binding to one or more target molecules in a complex mixture, the method including the steps of:
(a) providing a pool of candidate nucleic acid ligands;
(b) providing a first pool of target molecules;
(c) providing a second pool of target molecules, wherein the second pool of target molecules may be isolated from the first pool of target molecules, and wherein the second pool of target molecules differs from the first pool of target molecules in that one or more of the target molecules present in the second pool is present at a higher concentration than that present in the first pool of target molecules;
(d) allowing the nucleic acid ligands to bind to the first and second pools of target molecules, wherein the first and second pool of target molecules are in the presence of one another;
(e) isolating the nucleic acid ligands bound to the second pool of target molecules;
(f) amplifying the isolated nucleic acid ligands bound to the second pool of target molecules;
(g) reiterating steps (a) through (f) using the amplified nucleic acid ligands as the pool of candidate nucleic acid ligands, wherein the steps are reiterated until a final pool of nucleic acid ligands is obtained with a desired level of binding specificity to the second pool of target molecules; and
(h) isolating the final pool of nucleic acid ligands so produced, wherein the final pool of nucleic acid ligands allows the differentiation of a test pool of molecules from a control pool of molecules.

2. A method according to claim 1, wherein the candidate nucleic acid ligands are DNA ligands.

3. A method according to claim 1, wherein the first pool of target molecules is derived from a cellular extract.

4. A method according to claim 3, wherein the cellular extract is derived from colorectal tissue, breast tissue, cervical tissue, uterine tissue, renal tissue, pancreatic tissue, oesophageal tissue, stomach tissue, lung tissue, brain tissue, liver tissue, bladder tissue, bone tissue, prostate tissue, skin tissue, ovary tissue, testicular tissue, muscle tissue and vascular tissue.

5. A method according to claim 3, wherein the cellular extract is formalin fixed tissue.

6. A method according to claim 1, wherein the second pool of target molecules is derived from a cellular extract.

7. A method according to claim 6, wherein the cellular extract is derived from colorectal tissue, breast tissue, cervical tissue, uterine tissue, renal tissue, pancreatic tissue, oesophageal tissue, stomach tissue, lung tissue, brain tissue, liver tissue, bladder tissue, bone tissue, prostate tissue, skin tissue, ovary tissue, testicular tissue, muscle tissue and vascular tissue.

8. A method according to claim 6, wherein the cellular extract is formalin fixed tissue.

9. A method according to claim 1, wherein the first pool of target molecules is derived from a cellular extract from non-cancerous cells and the second pool of target cells is derived from a cellular extract from cancerous cells.

10. A method according to claim 1, wherein the test pool of molecules is derived from a cellular extract.

11. A method according to claim 10, wherein the cellular extract is derived from colorectal tissue, breast tissue, cervical tissue, uterine tissue, renal tissue, pancreatic tissue, oesophageal tissue, stomach tissue, lung tissue, brain tissue, liver tissue, bladder tissue, bone tissue, prostate tissue, skin tissue, ovary tissue, testicular tissue, muscle tissue and vascular tissue.

12. A method according claim 10, wherein the cellular extract is formalin fixed tissue.

13. A method according to claim 1, wherein the control pool of target molecules is derived from a cellular extract.

14. A method according to claim 13, wherein the cellular extract is derived from colorectal tissue, breast tissue, cervical tissue, uterine tissue, renal tissue, pancreatic tissue, oesophageal tissue, stomach tissue, lung tissue, brain tissue, liver tissue, bladder tissue, bone tissue, prostate tissue, skin tissue, ovary tissue, testicular tissue, muscle tissue and vascular tissue.

15. A method according to claim 13, wherein the cellular extract is formalin fixed tissue.

16. A method according to claim 1, wherein the control pool of molecules is derived from a cellular extract from non-cancerous cells and the test pool of molecules is derived from a cellular extract from cancerous cells.

17. A method according to claim 1, wherein amplification is by polymerase chain reaction or rolling circle replication.

18. A pool of nucleic acid ligands produced according to the method of claim 1.

19. A nucleic acid ligand isolated from the pool of nucleic acid ligands according to claim 18.

20. A method for isolating a plurality of individual nucleic acid ligands capable of binding to a plurality of different target molecules in a complex mixture of molecules, the method including the steps of:
(a) providing a pool of candidate nucleic acid ligands;
(b) providing a pool of target molecules, wherein the target molecules in the pool may be isolated;
(c) allowing the nucleic acid ligands to bind to the target molecules;
(d) isolating the nucleic acid ligands bound to the pool of target molecules;
(e) amplifying the isolated nucleic acid ligands;
(f) isolating an individual nucleic acid ligand from the amplified nucleic acid ligands;
(g) using the individual nucleic acid ligand to deplete the pool of target molecules of a specific molecule;
(h) reiterating steps (a) to (g) using the successively depleted pool of target molecules as the starting pool of target molecules for each cycle of reiteration, wherein the steps are reiterated until a plurality of individual nucleic acid ligands is identified.

21. A method according to claim 20, wherein the candidate nucleic acid ligands are DNA ligands.

22. A method according to claim 20, wherein the target molecules are derived from a cellular extract.

23. A method according to claim 20, wherein the cellular extract is derived from colorectal tissue, breast tissue, cervical tissue, uterine tissue, renal tissue, pancreatic tissue, oesophageal tissue, stomach tissue, lung tissue, brain tissue, liver tissue, bladder tissue, bone tissue, prostate tissue, skin tissue, ovary tissue, testicular tissue, muscle tissue and vascular tissue.

24. A method according to claim 20, wherein amplification is by polymerase chain reaction or rolling circle replication.

25. A method according to claim 20, wherein the depletion is by affinity chromatography.

26. A plurality of nucleic acid ligands produced according to the method of 20.

27. A nucleic acid ligand isolated from the plurality of nucleic acid ligands according to claim 26.

28. A method for isolating a nucleic acid ligand capable of binding to a target molecule in a complex mixture, the method including the steps of:
(a) providing a pool of candidate nucleic acid ligands;
(b) providing a first pool of target molecules;
(c) providing a second pool of target molecules, wherein the second pool of target molecules may be isolated from the first pool of target molecules, and wherein the second pool of target molecules differs from the first pool of target molecules in that one or more of the target molecules present in the second pool is present at a higher concentration than that present in the first pool of target molecules;
(d) allowing the nucleic acid ligands to bind to the first and second pools of target molecules, wherein the first and second pool of target molecules are in the presence of one another;
(e) isolating the nucleic acid ligands bound to the second pool of target molecules;
(f) amplifying the isolated nucleic acid ligands bound to the second pool of target molecules;
(g) reiterating steps (a) through (f) using the amplified nucleic acid ligands as the pool of candidate nucleic acid ligands, wherein the steps are reiterated until a final pool of nucleic acid ligands is obtained with a desired level of binding specificity to the second pool of target molecules;
(h) isolating the final pool of nucleic acid ligands so produced, wherein the final pool of nucleic acid ligands allows the differentiation of a test pool of molecules from a control pool of molecules; and
(i) isolating a nucleic acid ligand from the final pool of nucleic acid ligands, wherein the isolated nucleic acid ligand is capable of binding to a target molecule in a complex mixture.

29. A method according to claim 28, wherein the candidate nucleic acid ligands are DNA ligands.

30. A method according to claim 28, wherein the first pool of target molecules is derived from a cellular extract.

31. A method according to claim 30, wherein the cellular extract is derived from colorectal tissue, breast tissue, cervical tissue, uterine tissue, renal tissue, pancreatic tissue, oesophageal tissue, stomach tissue, lung tissue, brain tissue, liver tissue, bladder tissue, bone tissue, prostate tissue, skin tissue, ovary tissue, testicular tissue, muscle tissue and vascular tissue.

32. A method according to claim 30, wherein the cellular extract is formalin fixed tissue.

33. A method according to claim 28, wherein the second pool of target molecules is derived from a cellular extract.

34. A method according to claim 33, wherein the cellular extract is derived from colorectal tissue, breast tissue, cervical tissue, uterine tissue, renal tissue, pancreatic tissue, oesophageal tissue, stomach tissue, lung tissue, brain tissue, liver tissue, bladder tissue, bone tissue, prostate tissue, skin tissue, ovary tissue, testicular tissue, muscle tissue and vascular tissue.

35. A method according to claim 35, wherein the cellular extract is formalin fixed tissue.

36. A method according to claim 28, wherein the first pool of target molecules is derived from a cellular extract from non-cancerous cells and the second pool of target cells is derived from a cellular extract from cancerous cells.

37. A method according to claim 28, wherein the test pool of molecules is derived from a cellular extract.

38. A method according to claim 37, wherein the cellular extract is derived from colorectal tissue, breast tissue, cervical tissue, uterine tissue, renal tissue, pancreatic tissue, oesophageal tissue, stomach tissue, lung tissue, brain tissue, liver tissue, bladder tissue, bone tissue, prostate tissue, skin tissue, ovary tissue, testicular tissue, muscle tissue and vascular tissue.

39. A method according to claim 37, wherein the cellular extract is formalin fixed tissue.

40. A method according to claim 28, wherein the control pool of target molecules is derived from a cellular extract.

41. A method according to claim 40, wherein the cellular extract is derived from colorectal tissue, breast tissue, cervical tissue, uterine tissue, renal tissue, pancreatic tissue, oesophageal tissue, stomach tissue, lung tissue, brain tissue, liver tissue, bladder tissue, bone tissue, prostate tissue, skin tissue, ovary tissue, testicular tissue, muscle tissue and vascular tissue.

42. A method according to claim 40, wherein the cellular extract is formalin fixed tissue.

43. A method according to claim 28, wherein the control pool of molecules is derived from a cellular extract from non-cancerous cells and the test pool of molecules is derived from a cellular extract from cancerous cells.

44. A method according to claim 28, wherein amplification is by polymerase chain reaction or rolling circle replication.

45. A nucleic acid ligand produced according to the method of claim 28.

46. A nucleic acid ligand according to claim 45, wherein the nucleic acid ligand allows the differentiation of a test pool of molecules from a control pool of molecules.

47. A polynucleotide including the nucleotide sequence according to SEQ ID NO:1.

48. A polynucleotide including a variant of the nucleotide sequence according to SEQ ID NO.1, wherein the polynucleotide forms a nucleic acid ligand that identifies at least one difference at the molecular level between two complex biological mixtures.

49. A polynucleotide according to claim 48, wherein the polynucleotide forms a nucleic acid ligand that identifies at least one difference at the molecular level between a malignant mesothelioma cell and a non-malignant mesothelioma cell.

50. A polynucleotide according to claim 48, wherein the polynucleotide forms a nucleic acid ligand that identifies at least one difference at the molecular level between a malignant lung cell and a non-malignant lung cell.

51. A polynucleotide according to claim 48, wherein the polynucleotide forms a nucleic acid ligand that identifies at least one difference at the molecular level between a malignant bowel cell and a non-malignant bowel cell.

52. A polynucleotide according to claim 48, wherein the polynucleotide forms a nucleic acid ligand that identifies at least one difference at the molecular level between a malignant prostate cell and a non-malignant prostate cell.

53. A polynucleotide that hybridises with the complement of the nucleotide sequence according to SEQ ID NO.1 under stringent hybridisation conditions, wherein the polynucleotide forms a nucleic acid ligand that identifies at least one difference at the molecular level between two complex biological mixtures.

54. A polynucleotide according to claim 53, wherein the stringent hybridisation conditions include hybridisation in 4×SSC at 65° C. and washing in 0.1×SSC at 65° C.

55. A polynucleotide according to claim 53, wherein the stringent hybridisation conditions include hybridisation in 50% formamide, 5×SSC and 1% SDS at 65° C. and washing in 0.2×SSC and 0.1% SDS at 65° C.

56. A nucleic acid ligand that distinguishes a malignant cell from a non-malignant cell.

57. A nucleic acid ligand according to claim 56, wherein the malignant cell is a malignant mesothelioma cell and the non-malignant cell is a non-malignant mesothelial cell.

58. A nucleic acid ligand according to claim 56, wherein the malignant cell is a malignant lung cell and the non-malignant cell is a non-malignant lung cell.

59. A nucleic acid ligand according to claim 56, wherein the malignant cell is a malignant bowel cell and the non-malignant cell is a non-malignant bowel cell.

60. A nucleic acid ligand according to claim 56, wherein the malignant cell is a malignant prostate cell and the non-malignant cell is a non-malignant prostate cell.

61. A nucleic acid ligand according to claim 57, wherein the nucleic acid ligand includes a nucleotide sequence according to SEQ ID NO:1 or a variant of the nucleotide sequence according to SEQ ID NO:1.

62. A method of identifying at least one difference at the molecular level between a first complex biological mixture and a second complex biological mixture, the method including the steps of:
(a) binding to a first complex biological mixture a nucleic acid ligand including the nucleotide sequence of SEQ ID NO:1 or a variant thereof;
(b) binding to a second complex biological mixture a nucleic acid ligand including the nucleotide sequence of SEQ ID NO:1 or a variant thereof; and
(c) identifying at least one difference at the molecular level between the first complex biological mixture and the second complex biological mixture by the differential binding of the nucleic acid ligand to the first complex biological mixture and the second biological mixture.

63. A method according to claim 62, wherein the first complex biological mixture is a malignant cell or extract thereof and the second complex biological system is a non-malignant cell or extract thereof.

64. A method according to claim 63, wherein the malignant cell is a mesothelioma cell and the non-malignant cell is a non-malignant mesothelial cell.

65. A method according to claim 63, wherein the malignant cell is a malignant lung cell and the non-malignant cell is a non-malignant lung cell.

66. A method according to claim 63, wherein the malignant cell is a malignant bowel cell and the non-malignant cell is a bowel cell.

67. A method according to claim 63, wherein the malignant cell is a malignant prostate cell and the non-malignant cell is a non-malignant prostate cell.

68. A method of identifying a malignant cell, the method including the steps of:
(a) binding to a test cell or cellular extract a nucleic acid ligand including the nucleotide sequence of SEQ ID NO:1 or a variant thereof;
(b) binding to a non-malignant cell or cellular extract a nucleic acid ligand including the nucleotide sequence of SEQ ID NO:1 or a variant thereof; and
(c) identifying the test cell as a malignant cell by differential binding of the nucleic acid ligand to the test cell or cellular extract and the non-malignant cell or cellular extract.

69. A method according to claim 68, wherein the malignant cell is a mesothelioma cell and the non-malignant cell is a non-malignant mesothelial cell.

70. A method according to claim 68, wherein the malignant cell is a malignant lung cell and the non-malignant cell is a non-malignant lung cell.

71. A method according to claim 68, wherein the malignant cell is a malignat bowel cell and the non-malignant cell is a non-malignant bowel cell.

72. A method according to claim 68, wherein the malignant cell is a malignant prostate cell and the non-malignant cell is a non-malignant prostate cell.


SEQUENCE LISTING (34 sequences)

SEQ ID NO: 1 - DNA, 68 bp, Artificial Sequence
  polynucleotide sequence of a nucleic acid ligand capable of distinguishing malignant mesothelioma cells from non malignant mesothelial cells.
gggagctcagaataaacgctcaaggaacagcaagatacggtcaccgaacatagcgcaccacaggcaca

SEQ ID NO: 2 - DNA, 68 bp, Artificial Sequence
  polynucleotide sequence of a nucleic acid ligand capable of distinguishing malignant mesothelioma cells from non malignant mesothelial cells. Variant of SEQ ID NO 1.
gggagctcagaataaacgctcaacaaaagactatccagcgacacgcaatctcaagcaacagaggacag

SEQ ID NO: 3 - DNA, 68 bp, Artificial Sequence
  polynucleotide sequence of a nucleic acid ligand capable of distinguishing malignant mesothelioma cells from non malignant mesothelial cells. Variant of SEQ ID NO 1.
gggagctcagaataaacgctcaagccatggacaagactaacgacagacctaaacctaaaggataaaaa

SEQ ID NO: 4 - DNA, 68 bp, Artificial Sequence
  polynucleotide sequence of a nucleic acid ligand capable of distinguishing malignant mesothelioma cells from non malignant mesothelial cells. Variant of SEQ ID NO 1.
gggagctcagaataaacgctcaaacccgaaaagcgggaaaacccccagcaaatcccgaccaaaagcaa

SEQ ID NO: 5 - DNA, 64 bp, Artificial Sequence
  polynucleotide sequence of a nucleic acid ligand capable of distinguishing malignant mesothelioma cells from non malignant mesothelial cells. Variant of SEQ ID NO 1.
gggagctcagaataaacgctcaacctgttttttttcccccttattcttcccccccctgtgtcgc

SEQ ID NO: 6 - DNA, 68 bp, Artificial Sequence
  polynucleotide sequence of a nucleic acid ligand capable of distinguishing malignant mesothelioma cells from non malignant mesothelial cells. Variant of SEQ ID NO 1.
gggagctcagaataaacgctcaattgtgtgtcttcttgctcttcttccttcccttgccttgccattgt

SEQ ID NO: 7 - DNA, 68 bp, Artificial Sequence
  polynucleotide sequence of a nucleic acid ligand capable of distinguishing malignant mesothelioma cells from non malignant mesothelial cells. Variant of SEQ ID NO 1.
gggagctcagaataaacgctcaatgtgtgctgtcaggcgtgctgtgtgtgtaatcttggtgcggcctc

SEQ ID NO: 8 - DNA, 67 bp, Artificial Sequence
  polynucleotide sequence of a nucleic acid ligand capable of distinguishing malignant mesothelioma cells from non malignant mesothelial cells. Variant of SEQ ID NO 1.
gggagctcagaataaacgctcaagcaggaccaagaagacacccaaaaagaagcaatgaaagaggcag

SEQ ID NO: 9 - DNA, 68 bp, Artificial Sequence
  polynucleotide sequence of a nucleic acid ligand capable of distinguishing malignant mesothelioma cells from non malignant mesothelial cells. Variant of SEQ ID NO 1.
gggagctcagaataaacgctcaagttggggtgttgcctggtctgtgtacgctgggggcggttgcgtgg

SEQ ID NO: 10 - DNA, 63 bp, Artificial Sequence
  polynucleotide useful for distinguishing malignant prostate cells from non-malignant prostate cells.
gggagctcagaataaacgctcaacaattttctttttcccttctctgtccttttctccgtcttg

SEQ ID NO: 11 - DNA, 64 bp, Artificial Sequence
  polynucleotide useful for distinguishing malignant prostate cells from non-malignant prostate cells.
gggagctcagaataaacgctcaacaattttctttttcccttctctgtccttttctccgtgcttg

SEQ ID NO: 12 - DNA, 68 bp, Artificial Sequence
  polynucleotide useful for distinguishing malignant prostate cells from non-malignant prostate cells.
gggagctcagaataaacgctcaagtttttcctcctgcctgttttcttccccgtgctccttttcccccc

SEQ ID NO: 13 - DNA, 69 bp, Artificial Sequence
  polynucleotide useful for distinguishing malignant prostate cells from non-malignant prostate cells.
gggagctcagaataaacgctcaaaagaatcagcagagacagggaggcgagaaagaagggggggggggag

SEQ ID NO: 14 - DNA, 68 bp, Artificial Sequence
  polynucleotide useful for distinguishing malignant prostate cells from non-malignant prostate cells.
gggagctcagaataaacgctcaacagccaggacagagaggtgggaaacccaaacaagagcaaatagcc

SEQ ID NO: 15 - DNA, 68 bp, Artificial Sequence
  polynucleotide useful for distinguishing malignant prostate cells from non-malignant prostate cells.
gggagctcagaataaacgctcaattgttttggctttgtctcccgtttgccttccccggcctttgtctg

SEQ ID NO: 16 - DNA, 68 bp, Artificial Sequence
  polynucleotide useful for distinguishing malignant prostate cells from non-malignant prostate cells.
gggagctcagaataaacgctcaatctgggtctgtgtgtatcttttccattgcctccttcccttcgtct

SEQ ID NO: 17 - DNA, 68 bp, Artificial Sequence
  polynucleotide useful for distinguishing malignant prostate cells from non-malignant prostate cells.
gggagctcagaataaacgctcaatcttgccggttctctcctttttcctgtctgccttctttctccttg

SEQ ID NO: 18 - DNA, 67 bp, Artificial Sequence
  polynucleotide useful for distinguishing malignant prostate cells from non-malignant prostate cells.
gggagctcagaataaacgctcaacctcctgtctgctcctatctcttgccttccttgtttcccctgcc

SEQ ID NO: 19 - DNA, 68 bp, Artificial Sequence
  polynucleotide useful for distinguishing malignant prostate cells from non-malignant prostate cells.
gggagctcagaataaacgctcaactctgtcctttccctttctccctttcttgctgctcctttcgtgtc

SEQ ID NO: 20 - DNA, 68 bp, Artificial Sequence
  polynucleotide useful for distinguishing malignant prostate cells from non-malignant prostate cells.
gggagctcagaataaacgctcaacacttttcttgtccatttgcttctctaccctcattctcccatcct

SEQ ID NO: 21 - DNA, 68 bp, Artificial Sequence
  polynucleotide useful for distinguishing malignant prostate cells from non-malignant prostate cells.
gggagctcagaataaacgctcaaagcctctctaccgtggtgctgcccttcgatttgtgtctgctgtgt

SEQ ID NO: 22 - DNA, 67 bp, Artificial Sequence
  polynucleotide useful for distinguishing malignant prostate cells from non-malignant prostate cells.
gggagctcagaataaacgctcaatgtggttttgccttttctttccgttttctctttccctcgcgcct

SEQ ID NO: 23 - DNA, 68 bp, Artificial Sequence
  polynucleotide useful for distinguishing malignant prostate cells from non-malignant prostate cells.
gggagctcagaataaacgctcaaccccctgcttccccctccttatgttgtcctgccggcgcctgtact

SEQ ID NO: 24 - DNA, 68 bp, Artificial Sequence
  polynucleotide useful for distinguishing malignant prostate cells from non-malignant prostate cells.
gggagctcagaataaacgctcaatcactgtcgtcatttattttttcagtcctatttccctctcctgtg

SEQ ID NO: 25 - DNA, 68 bp, Artificial Sequence
  polynucleotide useful for distinguishing malignant prostate cells from non-malignant prostate cells.
gggagctcagaataaacgctcaatcctcttttttgtacggcctgctgtttgtctgtgtgtcttcctca

SEQ ID NO: 26 - DNA, 68 bp, Artificial Sequence
  polynucleotide useful for distinguishing malignant prostate cells from non-malignant prostate cells.
gggagctcagaataaacgctcaaccagtcggctcctttcctgcgcgtctcttcccgttttttccccct

SEQ ID NO: 27 - DNA, 68 bp, Artificial Sequence
  polynucleotide useful for distinguishing malignant prostate cells from non-malignant prostate cells.
gggagctcagaataaacgctcaatgttgcctaattcctgctatgtttctgtctcctccccacccgcgc

SEQ ID NO: 28 - DNA, 68 bp, Artificial Sequence
  polynucleotide useful for distinguishing mali gnant adenoma cells from non-malignant bowel cells.
gggagctcagaataaacgctcaagcccccatagcagcaaagtaagaacaaccaacagacgcacgacgg

SEQ ID NO: 29 - DNA, 68 bp, Artificial Sequence
  polynucleotide useful for distinguishing mali gnant adenoma cells from non-malignant bowel cells.
gggagctcagaataaacgctcaaccaaaagaacacaacagaaccaagcagacacccacaccaccgcag

SEQ ID NO: 30 - DNA, 68 bp, Artificial Sequence
  polynucleotide useful for distinguishing mali gnant adenoma cells from non-malignant bowel cells.
gggagctcagaataaacgctcaaagttggtgttttcctttccctgtccccttgtttcatcttccctac

SEQ ID NO: 31 - DNA, 68 bp, Artificial Sequence
  polynucleotide useful for distinguishing mali gnant adenoma cells from non-malignant bowel cells.
gggagctcagaataaacgctcaatccctttttcccatcttttcgcggttgttgagctttctgcgtgtg

SEQ ID NO: 32 - DNA, 68 bp, Artificial Sequence
  polynucleotide useful for distinguishing mali gnant adenoma cells from non-malignant bowel cells.
gggagctcagaataaacgctcaaggaccagcacacacaccaacaaaggccagggacccggtacccacc

SEQ ID NO: 33 - DNA, 85 bp, Artificial Sequence
  Synthetic 85mer with a 45 base section of random nucleotides.
agctcagaataaacgctcaannnnnnnnnnnnnnnnnnnnnnnnnnnnnnnnnnnnnnnnnnnnnttcgacatgaggcccggatc

SEQ ID NO: 34 - DNA, 20 bp, Artificial Sequence
  Synthetic biotin labelled aptamer for use in screening.
gatccgggcctcatgtcgaa